(19) United States
(12) Patent Application Publication
(10) Pub. No.: US 2010/0144741 A1
(43) Pub. Date: Jun. 10, 2010

(54) ETHANOLAMINE DERIVATIVES USEFUL AS BACE INHIBITORS

(76) Inventors: Mathias Frederiksen, Basel (CH); Rainer Martin Luond, Therwil (CH); Clive McCarthy, Basel (CH); Henrik Moebitz, Freiburg (DE); Jean-Michel Rondeau, Rixheim (FR); Bernard Lucien Roy, Fribourg (CH); Heinrich Rueeger, Flueh (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/515,521

(22) PCT Filed: Nov. 22, 2007
(86) PCT No.: PCT/EP2007/062701

§ 371 Date: May 19, 2009

(30) Foreign Application Priority Data
Nov. 23, 2006 (EP) 06124689.8

(51) Int. Cl. A61K 31/4402; C07D 401/02; C07D 239/42; C07C 233/40; C07D 213/72; C07D 405/10; C07D 237/20; C07D 487/04; A61K 31/505; A61K 31/506; A61K 31/4418; A61K 31/50; A61K 31/5025; A61P 25/00

(52) U.S. Cl. 514/248; 544/328; 544/329; 564/220; 546/304; 546/312; 544/224; 544/236; 514/256; 514/352; 514/247

(57) ABSTRACT

The invention relates to novel cyclic compounds of the formula (I), in free base form or in acid addition salt form, to their preparation, to their use as medicaments and to medicaments comprising them.

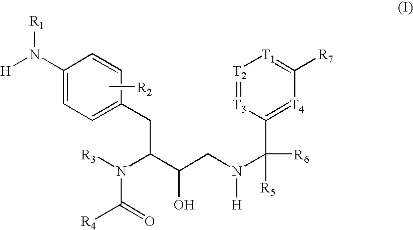

The present invention relates to novel cyclic compounds, to their preparation, to their use as medicaments and to medicaments comprising them.

More particularly the invention relates to a compound of the formula

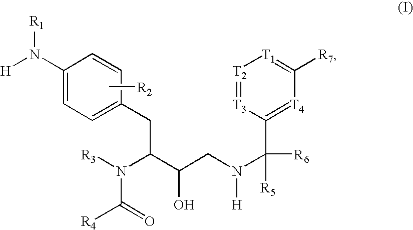

in which
R₁ is hydrogen, (C₁₋₈)alkyl, a (C₃₋₈)cycloalkyl, aryl or heteroaryl group, which (C₃₋₈)cycloalkyl, aryl or heteroaryl group is optionally substituted by 1 to 4 substituents, independently selected from the group consisting of halogen, (C₁₋₈)alkyl, halogen-substituted (C₁₋₈)alkyl, (C₁₋₈)alkoxy, (C₁₋₈)alkoxy(C₁₋₈)alkyl, (C₃₋₈)cycloalkyl and an aryl or heteroaryl group, which aryl or heteroaryl group is optionally substituted by 1 to 4 substituents, independently selected from the group consisting of halogen, (C₁₋₈)alkyl, halogen-substituted (C₁₋₈)alkyl, hydroxy, (C₁₋₈)alkoxy, (C₁₋₈)alkoxy(C₁₋₈)alkyl and (C₃₋₈)cycloalkyl, a group of the formula

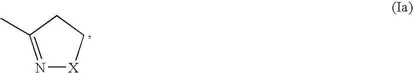

in which X is O or S, the group of the formula Ia being optionally substituted by 1 or 2 substituents, independently selected from the group consisting of halogen and (C₁₋₈)alkyl, or a group of the formula

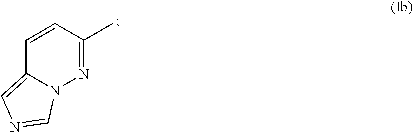

R₂ is hydrogen, halogen, (C₁₋₈)alkyl, (C₁₋₈)alkoxy, (C₁₋₈)alkoxy(C₁₋₈)alkyl, (C₁₋₈)alkylthio or a (C₃₋₈)cycloalkyl, (C₃₋₈)cycloalkyl(C₁₋₈)alkyl or (C₃₋₈)cycloalkyl(C₁₋₈)alkoxy group, in which (C₃₋₈)cycloalkyl, (C₃₋₈)cycloalkyl(C₁₋₈)alkyl or (C₃₋₈)cycloalkyl(C₁₋₈)alkoxy group the (C₃₋₈)cycloalkyl moiety is optionally substituted by 1 to 4 substituents, independently selected from the group consisting of halogen and (C₁₋₈)alkyl;
   either
R₃ is hydrogen and
R₄ is hydrogen, (C₁₋₈)alkyl, halogen-substituted (C₁₋₈)alkyl, (C₁₋₈)alkoxy(C₁₋₈)alkyl, (C₁₋₈)alkylthio(C₁₋₈)alkyl, (C₁₋₈)alkylamino(C₁₋₈)alkyl, a (C₃₋₈)cycloalkyl, aryl or heteroaryl group, which (C₃₋₈)cycloalkyl, aryl or heteroaryl group is optionally substituted by 1 to 4 substituents, independently selected from the group consisting of halogen, (C₁₋₈)alkyl, halogen-substituted (C₁₋₈)alkyl, (C₁₋₈)alkoxy, (C₁₋₈)alkoxy(C₁₋₈)alkyl, (C₃₋₈)cycloalkyl and an aryl or heteroaryl group, which aryl or heteroaryl group is optionally substituted by 1 to 4 substituents, independently selected from the group consisting of halogen, (C₁₋₈)-alkyl, halogen-substituted (C₁₋₈)alkyl, hydroxy, (C₁₋₈)alkoxy, (C₁₋₈)alkoxy(C₁₋₈)alkyl and (C₃₋₈)cycloalkyl, or a (C₃₋₈)cycloalkyl group, in which (C₃₋₈)cycloalkyl group one —CH₂-moiety is replaced by —O— and which (C₃₋₈)cycloalkyl group is optionally substituted by 1 or 2 substituents, independently selected from the group consisting of halogen and (C₁₋₈)alkyl,
   or
   the moiety —N(R₃)—C(═O)—R₄ is a group of the formula

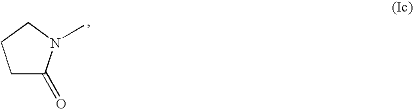

which is optionally substituted by 1 or 2 substituents, independently selected from the group consisting of halogen and (C₁₋₈)alkyl, or a group of the formula

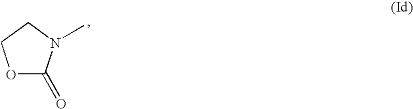

which is optionally substituted by 1 or 2 substituents, independently selected from the group consisting of halogen and (C₁₋₈)alkyl;
   either
R₅ is hydrogen, (C₁₋₈)alkyl, (C₁₋₈)alkoxy(C₁₋₈)alkyl or halogen-substituted (C₁₋₈)alkyl and
R₆ is hydrogen or (C₁₋₈)alkyl
   or
R₅ and R₆ together are, together with the carbon atom, to which they are attached, a (C₃₋₈)-cycloalkyl group, which (C₃₋₈)cycloalkyl group is unsubstituted or substituted by 1 to 4 substituents, independently selected from the group consisting of halogen and (C₁₋₈)alkyl;
R₇ is (C₁₋₈)alkyl, (C₃₋₈)cycloalkyl(C₁₋₈)alkyl or halogen-substituted (C₁₋₈)alkyl;
T₁ is CR₈, N, O, S or a bond;
R₆ is hydrogen, halogen, (C₁₋₈)alkyl, (C₁₋₈)alkoxy or halogen-substituted (C₁₋₈)alkyl;
T₂ is CR₉, N, O, S or a bond;
R₉ is hydrogen, halogen, (C₁₋₈)alkyl, (C₁₋₈)alkoxy or halogen-substituted (C₁₋₈)alkyl;
T₃ is CR₁₀, N, O, S or a bond;
R₁₀ is hydrogen, halogen, (C₁₋₈)alkyl, (C₁₋₈)alkoxy or halogen-substituted (C₁₋₈)alkyl;
T₄ is CR₁₁, N, O or S;
R₁₁ is hydrogen, halogen, (C₁₋₈)alkyl, (C₁₋₈)alkoxy or halogen-substituted (C₁₋₈)alkyl;
   the number of ring atoms included in the ring comprising T₁ being 5 or 6;
   the number of hetero ring atoms included in the ring comprising T₁ being 0, 1, 2 or 3; the hetero ring atoms optionally included in the ring comprising T₁ being selected, if the number of ring atoms included in the ring comprising T₁ is 5, in such a way, that any ring oxygen atom, which is optionally present, is separated from any other ring oxygen atom, which is optionally present, by at least one ring atom different from a ring oxygen atom; and the hetero ring atoms optionally included in the ring comprising T₁ being selected, if the number of ring atoms included in the ring comprising T₁ is 6, in such a way, that any ring oxygen atom, which is optionally present, is separated from any other ring hetero atom, which is optionally present, by at least one ring carbon atom, in free base form or in acid addition salt form.

On account of the asymmetrical carbon atoms present in the compounds of the formula I, the compounds may exist in pure optically active form or in the form of mixtures of optical isomers, e.g. in the form of racemic mixtures. All pure optical isomers and all their mixtures, including the racemic mixtures, are part of the present invention.

Halogen denotes fluorine, bromine, chlorine or iodine.

Aryl is naphthyl or, preferably, phenyl. It can also be fused with a cycloalkyl or a heteroaromatic ring (e.g. to form a quinolyl or indolyl group).

Heteroaryl is an aromatic 5- or 6-membered ring, in which 1, 2 or 3 ring atoms are hetero atoms independently selected from O, N and S, such as thiazolyl, oxazolyl or, preferably, pyridyl or pyrimidyl. It can also be fused with a cycloalkyl or an aromatic or heteroaromatic ring (e.g. to form a quinolyl or indolyl group).

Any non-cyclic carbon containing group or moiety with more than 1 carbon atom is straight-chain or branched.

Unless defined otherwise, carbon containing groups, moieties or molecules contain 1 to 8, preferably 1 to 6, more preferably 1 to 4, most preferably 1 or 2, carbon atoms.

In preferred embodiments, the invention relates to a compound of the formula I, in free base form or in acid addition salt form, in which

(1) R₂ is (C₁₋₈)alkyl, (C₁₋₈)alkoxy or, preferably, hydrogen;
(2) R₃ is hydrogen;
(3) R₄ is halogen-substituted (C₁₋₈)alkyl, (C₁₋₈)alkoxy(C₁₋₈)alkyl or, preferably, (C₁₋₈)alkyl;
(4) R₅ and R₆ together are, together with the carbon atom, to which they are attached, a (C₃₋₈)cycloalkyl group, which (C₃₋₈)cycloalkyl group is unsubstituted;
(5) R₇ is (C₁₋₈)alkyl;
(6) each of T₁, T₂, T₃ and T₄ is CH;
(7) each of T₁, T₂ and T₄ is CH and T₃ is N.

The preferred embodiments (1) to (7) are preferred independently, collectively or in any combination or sub-combination.

In especially preferred embodiments, the invention relates to one or more than one of the compounds of the formula I mentioned in the Examples hereinafter, in free base form or in acid addition salt form.

In a further aspect, the invention relates to a process for the preparation of the compounds of the formula I and their salts, comprising the steps of

a) reaction of a compound of the formula

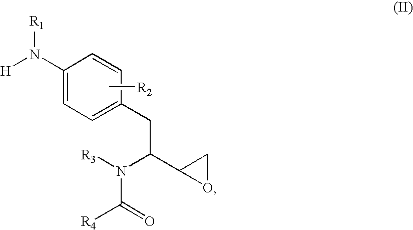

in which R₁, R₂, R₃ and R₄ are as defined for the formula I, with a compound of the formula

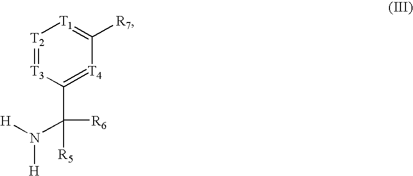

in which R₅, R₆, R₇, T₁, T₂, T₃ and T₄ are as defined for the formula I, or
b) reaction of a compound of the formula

R₁-L (IV),

in which R₁ is as defined for the formula I and L is a leaving group, with a compound of the formula

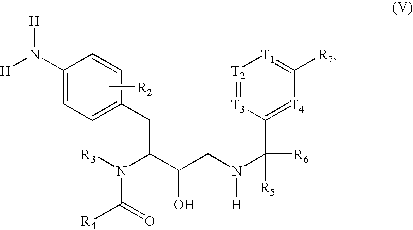

in which R₂, R₃, R₄, R₅, R₆, R₇, T₁, T₂, T₃ and T₄ are as defined for the formula I, or
c) for the preparation of a compound of the formula I, in which R₃ is hydrogen, reaction of a compound of the formula

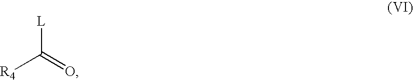

in which R₄ is as defined for the formula I and L is a leaving group, with a compound of the formula

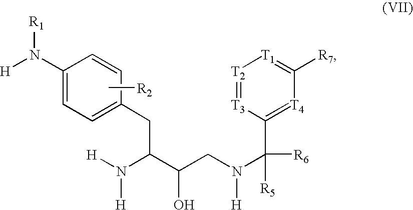

in which R₁, R₂, R₅, R₆, R₇, T₁, T₂, T₃ and T₄ are as defined for the formula I, or
d) for the preparation of a compound of the formula I, in which the moiety —N(R₃)—C(═O)—R₄ is 2-oxopyrrolidin-1-yl, intramolecular cyclisation of a compound of the formula

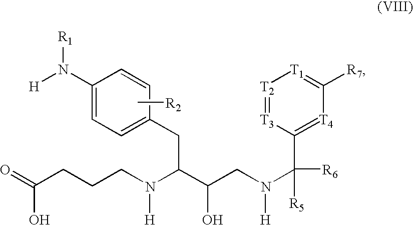

in which R₁, R₂, R₅, R₆, R₇, T₁, T₂, T₃ and T₄ are as defined for the formula I, or
in each case optionally followed by reduction, oxidation or other functionalisation of the resulting compound and/or by cleavage of any protecting group(s) optionally present, and of recovering the so obtainable compound of the formula I in free base form or in acid addition salt form.

The reactions can be effected according to conventional methods, for example as described in the Examples.

The working-up of the reaction mixtures and the purification of the compounds thus obtainable may be carried out in accordance with known procedures.

Acid addition salts may be produced from the free bases in known manner, and vice-versa.

Compounds of the formula I can also be prepared by further conventional processes, which processes are further aspects of the invention, e.g. as described in the Examples.

The starting materials of the formulae II, III, IV, V, VI, VII and VIII are known or may be prepared according to conventional procedures starting from known compounds, for example as described in the Examples.

Compounds of the formula I and their pharmaceutically acceptable acid addition salts, hereinafter referred to as “agents of the invention”, exhibit valuable pharmacological properties when tested in vitro and in animals, and are therefore useful as medicaments.

The agents of the invention are inhibitors of aspartic proteases and can be used for the treatment of disorders involving processing by such enzymes. Particularly they inhibit beta-secretase and as such inhibit the generation of beta-amyloid and the subsequent aggregation into oligomers and fibrils.

### Test 1: Inhibition of Human BACE

Recombinant BACE (extracellular domain, expressed in baculovirus and purified using standard methods) at 0.1-10 nM concentration is incubated with the test compound at various concentrations for 1 hour at room temperature in 10-100 mM acetate buffer, pH 4.5, containing 0.1% CHAPS. Synthetic fluorescence-quenched peptide substrate, derived from the sequence of APP and containing a suitable fluorophore-quencher pair is added to a final concentration of 1-5 μM and the increase in fluorescence is recorded at a suitable excitation/emission wavelength in a microplate spectro-fluorimeter for 5-30 minutes in 1-minute intervals. IC₅₀ values are calculated from percentage of inhibition of BACE-activity as a function of the test compound concentration.

### Test 2: Inhibition of Human BACE-2

Recombinant BACE-2 (extracellular domain, expressed in baculovirus and purified using standard methods) at 0.1-10 nM concentrations is incubated with the test compound at various concentrations for 1 hour at room temperature in 10-100 mM acetate buffer, pH 4.5, containing 0.1% CHAPS. Synthetic peptide substrate derived from the sequence of APP and containing a suitable fluorophore-quencher pair is added to a final concentration of 1-5 μM and the increase in fluorescence is recorded at a suitable excitation/emission wavelength in a microplate spectro-fluorimeter for 5-30 minutes in 1-minute intervals. IC₅₀ values are calculated from percentage of inhibition of BACE-2-activity as a function of the test compound concentration.

### Test 3: Inhibition of Human Cathepsin D

Recombinant cathepsin D (expressed as procathepsin D in baculovirus, purified using standard methods and activated by incubation in sodium formate buffer pH 3.7) is incubated with the test compound at various concentrations for 1 hour at room temperature in sodium formate or sodium acetate buffer at a suitable pH within the range of pH 3.0-5.0 Synthetic peptide substrate Mca-Gly-Lys-Pro-Ile-Leu-Phe-Phe-Arg-Leu-Lys(DNP)-D-Arg-NH₂ is added to a final concentration of 1-5 μM and the increase in fluorescence is recorded at excitation of 325 nm and emission at 400 nm in a microplate spectro-fluorimeter for 5-30 minutes in 1-minute intervals. IC₅₀ values are calculated from percentage of inhibition of cathepsin D-activity as a function of the test compound concentration.

### Test 4: Inhibition of Cellular Release of Amyloid Peptide 1-40

Chinese hamster ovary cells are transfected with the gene for amyloid precursor protein. Cells are plated at a density of 8000 cells/well in a 96-well microtiter plate and cultivated for 24 hours in DMEM cell culture medium containing 10% FCS. The test compound is added to the cells at various concentrations, and cells are cultivated for 24 hours in the presence of the test compound. The supernatants are collected, and the concentration of amyloid peptide 1-40 is determined using sandwich ELISA. The potency of the compound is calculated from the percentage of inhibition of amyloid peptide release as a function of the test compound concentration.

In at least one of the above-indicated tests, the agents of the invention show activity at concentrations below 50 μM.

Specifically, the agent of the invention described in Example 2 shows an IC₅₀ value of 23 μM in Test 1.

The agents of the invention are therefore useful e.g. for the treatment and/or prevention of neurological and vascular disorders related to beta-amyloid generation and/or aggregation, such as neurodegenerative diseases like Alzheimer's disease, Down's Syndrome, memory and cognitive impairment, dementia, amyloid neuropathies, brain inflammation, nerve and brain trauma, vascular amyloidosis, or cerebral haemorrhage with amyloidosis.

Some of the agents of the invention also inhibit BACE2 (beta-site APP-cleaving enzyme 2) or Cathepsin D, close homologues of the pepsin-type aspartyl proteases and of beta-secretase. Due to the correlation of BACE2 and CathD expression with a more tumorigenic and metastatic potential of tumor cells, such inhibitors are useful for the suppression of the metastasis process associated with tumor cells.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range from about 10 to about 2000, preferably from about 10 to about 200, mg of an agent of the invention conveniently administered, for example, in divided doses up to four times a day or in sustained release form.

The agent of the invention may be administered by any conventional route, in particular enterally, preferably orally, for example in the form of tablets or capsules, or parenterally, for example in the form of injectable solutions or suspensions.

In accordance with the foregoing, the present invention also provides an agent of the invention, for use as a medicament, e.g. for the treatment of neurological or vascular disorders related to beta-amyloid generation and/or aggregation.

The present invention furthermore provides a pharmaceutical composition comprising an agent of the invention in association with at least one pharmaceutical carrier or diluent. Such compositions may be manufactured in conventional manner. Unit dosage forms contain, for example, from about 1 to about 1000, preferably from about 1 to about 500, mg of an agent of the invention.

The agents of the invention can be administered alone or in combination with other pharmaceutical agents effective in the treatment of conditions mentioned above.

The pharmaceutical combination may be in the form of a unit dosage form, whereby each unit dosage will comprise a predetermined amount of the two components, in admixture with suitable pharmaceutical carriers or diluents. Alternatively, the combination may be in form of a package containing the two components separately, e.g. a pack or dispenser-device adapted for the concomitant or separate administration of the two active agents, wherein these agents are separately arranged.

Moreover the present invention provides the use of an agent of the invention, for the manufacture of a medicament for the treatment of any neurological or vascular disorders related to beta-amyloid generation and/or aggregation.

In still a further aspect, the present invention provides a method for the treatment of any neurological or vascular disorders related to beta-amyloid generation and/or aggregation, in a subject in need of such treatment, which comprises administering to such subject a therapeutically effective amount of an agent of the invention.

The following Examples illustrate the invention, but do not limit it.

### EXAMPLES

### Abbreviations

ACN acetonitrile
AcOH acetic acid
Ac₂O acetic anhydride
Boc tert-butoxycarbonyl
BOP-Cl bis(2-oxo-3-oxazolidinyl)phosphonic chloride
CDI carbonyldiimidazole
DCE 1,2-dichloroethane
DCM dichloromethane
DIPEA diisopropyl-ethyl-amine

### DMAP 4-(N,N-dimethylamino)-pyridine

DMF dimethylformamide
DMSO dimethylsulfoxide
ESIMS electrospray ionization mass spectrometry
EtMgBr ethylmagnesium bromide
EtOAc ethyl acetate
EtOH ethanol
Et₂O diethyl ether
h hour(s)
HPLC high pressure liquid chromatography
iPrOH isopropanol
KOTMS potassium trimethylsilanolate
LDA lithium diisopropylamide
MeOH methanol
min minute(s)
MPLC medium pressure liquid chromatography
NEt₃ triethylamine
NMR nuclear magnetic resonance spectrometry
Pd₂(dba)₃ tris(dibenzylidene-acetone)dipaladium
P3P propylphosphonic anhydride
RT room temperature
TBME tert-butyl methyl ether
tBu tert-butyl
tBuOH tert-butanol
TFA trifluoroacetic acid
TFAA trifluoroacetic acid anhydride
THF tetrahydrofuran
TLC thin-layer chromatography
TMS trimethylsilyl
HPLC conditions (%=percent by volume)

### Method A (Rt_{A}=Retention Time A)

| | |
|---|---|
| Column type | SunFire C₁₈, 3.5 μm |
| Column dimension | 3.0 × 20 mm |
| Eluent | A) ACN |
| | B) Water + 0.1% TFA |
| Gradient | 5-95% A in 2.20 min + 0.50 min 95% A |
| Flow | 2.00 ml/min |

### Method B (Rt_{B}=Retention Time B)

| | |
|---|---|
| Column type | XTerra MS C₁₈, 2.5 μm |
| Column dimension | 50 × 2.1 mm |
| Eluent | A) ACN + 0.02% TFA |
| | B) Water + 0.02% TFA |
| Gradient | 10-95% A in 5.50 min + 2.10 min 90% A |
| Flow | 0.350 ml/min |

### Method C(Rt_{c}=Retention Time C)

| | |
|---|---|
| Column type | Nucleosil ® 5C₁₈, 3 μm |
| Column dimension | 50 × 5 mm |
| Eluent | A) Water + 0.1% TFA |
| | B) ACN + 0.1% TFA |
| Gradient | 10-100% B in 3 min + 1 min 100% B |
| Flow | 4 ml/min |

### Method D (Rt_{D}=Retention Time D)

| | |
|---|---|
| Column type | MN Nucleodur C18 Pyramid, 110 Angstroem, 5 μm |
| Column dimension | 125 × 4 mm |
| Eluent | A) Water + 0.1% TFA |
| | B) ACN + 0.1% TFA |
| Gradient | 5-100% B in 20 min |
| Flow | 1 ml/min |

### Method E (Rt_{E}=Retention Time E)

| | |
|---|---|
| Column type | XTerra C₁₈, 2.5 μm |
| Column dimension | 3 × 30 mm |
| Eluent | A) Water/5% ACN/0.2% HCOOH |
| | B) ACN/0.2% HCOOH |
| Gradient | 10-95% B in 2.5 min + 2.2 min 90% A |
| Flow | 0.7 ml/min |

### Method F (Rt_{F}=Retention Time F)

| | |
|---|---|
| Column type | Acquity BEH Shield RP18, 1.7 μm |
| Column dimension | 2.1 × 50 mm |
| Eluent | A) Water/3 mM ammonium acetate/0.05% HCOOH |
| | B) ACN/0.05% HCOOH |
| Gradient | 2-98% B in 5.5 min + 0.5 min 98% A |
| Flow | 0.6 ml/min |

### Example 1

### N-{(1S,2R)-3-[1-(4-tert-Butyl-pyridin-2-yl)-cyclopropylamino]-2-hydroxy-1-[4-(6-phenyl-pyrimidin-4-ylamino)-benzyl]-propyl}-acetamide

### a) (S)-2-tert-Butoxycarbonylamino-3-(4-nitro-phenyl)-propionic acid methyl ester

(S)-2-tert-Butoxycarbonylamino-3-(4-nitro-phenyl)-propionic acid (15.0 g, 48.4 mmol) is dissolved in MeOH (150 ml) and toluene (750 ml), and the solution is cooled to 0° C. TMS diazomethane (36 ml, 2 M in Et₂O, 73 mmol) is added slowly over 5 min. Then the reaction mixture is allowed to warm to RT. After stirring for 1 h, the solvent is removed, and (S)-2-tert-butoxycarbonylamino-3-(4-nitro-phenyl)-propionic acid methyl ester is obtained as a colourless solid [ESIMS [M-Boc+H]⁺=225; HPLC Rt_{A}=1.6 min].

### b) [(S)-3-Chloro-1-(4-nitro-benzyl)-2-oxo-propyl]-carbamic acid tert-butyl ester

Chloroiodomethane (6.92 ml, 92 mmol) is added to a stirred solution of (S)-2-tert-butoxycarbonylamino-3-(4-nitro-phenyl)-propionic acid methyl ester (7.5 g, 23.1 mmol) in THF (225 ml) at −78° C. under N₂. LDA (73.6 ml, 1.57 M in heptane/THF/ethylbenzene) is added drop-wise, while the temperature of the reaction mixture is maintained below −73° C. The mixture is stirred for 0.5 h and then carefully quenched with AcOH (34.8 ml), while the temperature is maintained below −65° C. After 15 min of stirring at −78° C., the mixture is warmed to 0° C., and half-saturated aqueous NaCl solution (100 ml) is added. The mixture is extracted with TBME (2×200 ml), and the organic layers are combined, washed with 1 M sodium sulfite solution and water, dried with Na₂SO₄ and evaporated to afford the title compound as a brown solid (used as such in the next reaction step) [ESIMS [M-Boc+H]⁺=243, 245; HPLC Rt_{A}=1.7 min].

### c) [(1S,2S)-3-Chloro-2-hydroxy-1-(4-nitro-benzyl)-propyl]-carbamic acid tert-butyl ester

2 equivalents of NaBH₄ are suspended in EtOH (150 ml), and the suspension is cooled to −78° C. A solution of [(S)-3-chloro-1-(4-nitro-benzyl)-2-oxo-propyl]-carbamic acid tert-butyl ester (17.73 g, purity 44.7%, 23.1 mmol) in EtOH (350 ml) is added dropwise, while maintaining the temperature below −75° C. The reaction mixture is stirred for 1 h at −78° C., then quenched with 1 N HCl being added dropwise and warmed to RT. The EtOH is removed, and the residual aqueous solution is extracted with EtOAc (2×200 ml). The combined organic layers are washed with half-saturated NaCl solution, dried with Na₂SO₄, filtered and concentrated. The residue is recrystallized from MeOH to give the title compound as a colourless solid [ESIMS [M−H]⁺=343, 345; HPLC Rt_{A}=1.5 min].

### d) [(1S,2S)-1-(4-Amino-benzyl)-3-chloro-2-hydroxy-propyl]-carbamic acid tert-butyl ester

A mixture of [(1S,2S)-3-chloro-2-hydroxy-1-(4-nitro-benzyl)-propyl]-carbamic acid tert-butyl ester (3.79 g, 11.0 mmol) and Pd on charcoal (10%, 1.20 g) in MeOH (100 ml) is stirred at 25° C. for 3 h under hydrogen. The palladium is filtered through Celite, and the solvent is removed in vacuo. The resulting solid is purified by MPLC with DCM/MeOH and 1% of NEt₃ to give the title compound as a yellow solid [ESIMS [M+Na]⁺=337, 339; HPLC Rt_{A}=0.8 min].

### e) (2S,3S)-3-Amino-1-chloro-4-[4-(6-phenyl-pyrimidin-4-ylamino)-phenyl]-butan-2-ol

A mixture of [(1S,2S)-1-(4-amino-benzyl)-3-chloro-2-hydroxy-propyl]carbamic acid tert-butyl ester (2.44 g, 7.75 mmol), aqueous 1 N HCl (13 ml, 13 mmol) and 4-chloro-6-phenyl-pyrimidine (3.68 g, 19.3 mmol) in iPrOH (22 ml) is microwaved with stirring at 150° C. for 10 min. The solvent is removed, and the residue is triturated with water. The resulting yellow precipitate is filtered off and purified by MPLC with DCM/MeOH and 1% of NEt₃ to give the title compound as a yellow solid [ESIMS [M+H]⁺=369, 371; HPLC Rt_{A}=0.9 min].

### f) N-{(1S,2S)-3-Chloro-2-hydroxy-1-[4-(6-phenyl-pyrimidin-4-ylamino)-benzyl]-propyl}-acetamide

Ac₂O (1.02 ml, 10.7 mmol) is added to a solution of (2S,3S)-3-amino-1-chloro-4-[4-(6-phenyl-pyrimidin-4-ylamino)-phenyl]-butan-2-ol (3.29 g, 8.92 mmol) in pyridine (40 ml), and the reaction mixture is stirred at 25° C. for 0.5 h. The solvent is removed, and the residue is taken up in DCM/MeOH (9:1). The mixture is washed with 1N HCl and brine. The organic layer is concentrated to give the title compound as a yellow solid [ESIMS [M+H]⁺=411, 413; HPLC Rt_{A}=0.9 min].

### g) N-{(S)-1-(S)-Oxiranyl-2-[4-(6-phenyl-pyrimidin-4-ylamino)-phenyl]-ethyl}-acetamide

1 M KOH (6.7 ml, 6.7 mmol) is added to a stirred solution of N-{(1S,2S)-3-chloro-2-hydroxy-1-[4-(6-phenyl-pyrimidin-4-ylamino)-benzyl]-propyl}-acetamide (1.38 g, 3.36 mmol) in MeOH (5.5 ml) and THF (5.5 ml). The mixture is stirred at 0° C. for 3 h and then quenched with brine (20 ml). The resulting precipitate is filtered off to give the title compound as a colourless solid [ESIMS [M+H]⁺=375; HPLC Rt_{A}=0.9 min].

### h) 1-(4-tert-Butyl-pyridin-2-yl)-cyclopropylamine

The title compound is prepared from 4-tert-butyl-pyridine-2-carbonitrile following the procedure of P. Bertus et al., J. Org. Chem. 2003, 68, 7133, or of A. de Meijere et al., Org. Lett. 2003, 5, 753 [TLC (CH₂Cl₂/MeOH/NH₃ 90:9:1) R_{f}=0.30; ESIMS [M+H]⁺=191; ¹H-NMR (400 MHz, DMSO-d₆) 8.26 (d, 1H), 7.77 (d, 1H), 7.08 (dd, 1H), 1.29 (s, 9H), 1.21-1.16 (m, 2H), 0.95-0.91 (m, 2H)].

### i) N-{(1S,2R)-3-[1-(4-tert-Butyl-pyridin-2-yl)-cyclopropylamino]-2-hydroxy-1-[4-(6-phenyl-pyrimidin-4-ylamino)-benzyl]-propyl}-acetamide

To 1-(4-tert-butyl-pyridin-2-yl)-cyclopropylamine (0.814 g, 4.26 mmol) is added N-{(S)-1-(S)-oxiranyl-2-[4-(6-phenyl-pyrimidin-4-ylamino)-phenyl]-ethyl}-acetamide (300 mg, 0.8 mmol), and the mixture is stirred under N₂ with a few drops of DMF at 80° C. for 30 h. The reaction mixture is then concentrated, and the residue is purified by preparative HPLC (ACN/water). The fractions containing the desired product are combined, and the ACN is removed. The aqueous phase is neutralized with 1 N NaOH and extracted with EtOAc. The organic layer is washed with brine and dried with MgSO₄. The product remaining after the evaporation of the solvent is taken up in tBuOH, and the mixture is freeze-dried to give the title compound as a colourless solid [ESIMS [M+H]⁺=565; Rt_{A}=1.1 min; ¹H-NMR (DMSO-d₆) 9.55 (s, 1H), 8.66 (s, 1H), 8.29 (d, 1H), 8.00 (d, 2H), 7.70 (m, 2H), 7.6-7.5 (m, 5H), 7.2-7.1 (m, 4H), 4.90 (d, 1H), 3.86 (m, 1H), 3.45 (m, 1H), 2.94 (dd, 1H), 2.7-2.5 (m, 3H), 1.70 (s, 3H), 1.30 (s, 9H), 1.22 (m, 4H)].

### Example 2

### N-{(1S,2R)-2-Hydroxy-3-[1-(3-isopropyl-phenyl)-cyclopropylamino]-1-[4-(6-phenyl-pyrimidin-4-ylamino)-benzyl]-propyl}-acetamide

### a) 1-(3-Isopropyl-phenyl)-cyclopropylamine

3-Isopropyl-benzonitrile (42 g, 286 mmol) is dissolved in Et₂O (670 ml) under argon. Titanium(IV)-isopropoxide (90.4 g, 315 mmol) is added. The mixture is cooled to −70° C., and EtMgBr (3 M in Et₂O, 210 ml, 630 mmol) is added within 1 h. The mixture is warmed to 10° C., and BF₃×Et₂O (48%, 169 g, 573 mmol) is added. After 1 h, the reaction mixture is quenched with 400 ml of 1 N HCl, basified to pH 10 using 2 N NaOH and filtered over Hyflo Super Gel. The organic layer is dried over Na₂SO₄, filtered and concentrated. The residue is purified by column chromatography using DCM/MeOH (19:1) to give the title compound [¹H-NMR (400 MHz, CDCl₃) 7.32-7.28 (t, 1H), 7.23 (s, 1H), 7.19-7.10 (m, 2H), 3.0-2.9 (m, 1H), 1.96 (s, 2H), 1.33 (d, 6H), 1.12-1.09 (m, 2H), 1.09-1.02 (m, 2H)].

### b) N-{(1S,2R)-2-Hydroxy-3-[1-(3-isopropyl-phenyl)-cyclopropylamino]-1-[4-(6-phenyl-pyrimidin-4-ylamino)-benzyl]-propyl}-acetamide

A 4 M mixture of LiOH (0.05 ml, 0.20 mmol) in iPrOH (0.140 ml), N-{(S)-1-(S)-oxiranyl-2-[4-(6-phenyl-pyrimidin-4-ylamino)-phenyl]-ethyl}-acetamide (50 mg, 0.13 mmol) and 1-(3-isopropyl-phenyl)-cyclopropylamine hydrochloride (42.4 mg, 0.20 mmol) are heated to 80° C. for 4 h with shaking. The reaction mixture is cooled to 25° C., poured into 1 N HCl and extracted with EtOAc. The organic layer is washed with saturated aqueous NaHCO₃ solution and brine, dried, filtered and concentrated. The residue is purified by preparative HPLC (ACN/water) to give the title compound as a light yellow solid [ESIMS [M+H]⁺=550; HPLC Rt_{A}=1.1 min; ¹H-NMR (DMSO-d₆) 9.7 (s, 1H), 8.9 (s, 2H), 8.6 (s, 1H), 8.0 (d, 2H), 7.8 (d, 1H), 7.6-7.5 (m, 5H), 7.4-7.2 (m, 4H), 7.1 (s, 1H), 7.0 (d, 2H), 5.7 (s, 1H), 3.7 (m, 1H), 3.5 (m, 1H), 3.0-2.7 (m, 4H), 1.60 (s, 3H), 1.3 (m, 2H), 1.2 (m, 8H)].

### Example 3

### N-{(1S,2R)-3-[1-(3-tert-Butyl-phenyl)-cyclopropylamino]-2-hydroxy-1-[4-(6-phenyl-pyrimidin-4-ylamino)-benzyl]-propyl}-acetamide

### a) 1-(3-tert-Butyl-phenyl)-cyclopropylamine

The title compound is prepared from 3-tert-butyl-benzonitrile in a manner analogous to that described in Example 2a) [TLC (cyclohexane/EtOAc 50:50) R_{f}=0.20; ESIMS [M+H]⁺=190; ¹H-NMR (400 MHz, DMSO-d₆) 7.40-7.37 (m, 1H), 7.28-7.26 (m, 2H), 7.16-7.12 (m, 1H), 1.35 (s, 9H), 1.10-1.06 (m, 2H), 1.02-0.98 (m, 2H)].

### b) N-{(1S,2R)-3-[1-(3-tert-Butyl-phenyl)-cyclopropylamino]-2-hydroxy-1-[4-(6-phenyl-pyrimidin-4-ylamino)-benzyl]-propyl}-acetamide

A mixture of iPrOH (0.56 ml), N-{(S)-1-(S)-oxiranyl-2-[4-(6-phenyl-pyrimidin-4-ylamino)-phenyl]-ethyl}-acetamide (200 mg, 0.53 mmol) and 1-(3-tert-butyl-phenyl)-cyclopropylamine (217 mg, 0.20 mmol) is heated to 80° C. for 3 h with shaking. The mixture is cooled to 25° C. and purified by preparative HPLC (ACN/water) to give the title compound as a colourless solid [ESIMS [M+H]⁺=564; HPLC Rt_{A}=1.2 min; ¹H-NMR (DMSO-d₆) 9.6 (s, 1H), 8.7 (s, 1H), 8.0 (d, 2H), 7.7 (d, 1H), 7.6-7.5 (m, 5H), 7.4 (s, 1H), 7.2-7.1 (m, 4H), 7.0 (d, 1H), 4.8 (d, 1H), 3.9 (m, 1H), 3.5 (m, 1H), 2.9 (d, 1H), 2.6-2.5 (m, 3H), 1.70 (s, 3H), 1.3 (s, 9H), 1.2 (m, 4H)].

### Example 4

### N-{(1S,2R)-1-{4-[6-(4-Fluoro-phenyl)-pyrimidin-4-ylamino]-benzyl}-2-hydroxy-3-[1-(3-isopropyl-phenyl)-cyclopropylamino]-propyl}-acetamide

### a) [(1S,2R)-2-Hydroxy-3-[1-(3-isopropyl-phenyl)-cyclopropylamino]-1-(4-nitro-benzyl)-propyl]-carbamic acid tert-butyl ester

1-(3-Isopropyl-phenyl)-cyclopropylamine hydrochloride (1.00 g, 3.243 mmol) is suspended in iPrOH (5 ml). LiOH (1 ml of 4 M solution in water, 3.9 mmol) is added dropwise, and the clear mixture is stirred for 15 min. [(S)-2-(4-nitro-phenyl)-1-(S)-oxiranyl-ethyl]-carbamic acid tert-butyl ester (1.00 g, 3.24 mmol) is added in one portion, and the mixture is stirred at 90° C. for 2 h. The volatiles are removed in vacuo, and the residue is dissolved in EtOAc and 1 N HCl. The layers are separated, the organic portion is dried and concentrated, and the resulting material is used without further purification [HPLC Rt_{B}=4.06 min; ESIMS [M−H]⁺=484; ¹H-NMR (360 MHz, CDCl₃) 8.18 (d, 2H), 7.46 (d, 2H), 7.30-7.10 (m, 4H), 4.54 (d, 1H), 3.75 (m, 1H), 3.40 (m, 1H), 3.20 (d, 1H), 3.00-2.65 (m, 4H), 1.38-1.20 (m, 15H), 1.18-0.96 (m, 4H)].

### b) [(S)-1-{(R)-3-[1-(3-Isopropyl-phenyl)-cyclopropyl]-2-oxo-oxazolidin-5-yl}-2-(4-nitro-phenyl)-ethyl]-carbamic acid tert-butyl ester

[(1S,2R)-2-Hydroxy-3-[1-(3-isopropyl-phenyl)-cyclopropylamino]-1-(4-nitro-benzyl)-propyl]-carbamic acid tert-butyl ester (1.8 g, 3.72 mmol) is dissolved in DCE (43 ml). DIPEA (1.2 ml, 7.44 mmol), CDI (1.51 g, 9.3 mmol) and DMAP (45 mg, 0.37 mmol) are added, and the mixture is stirred at RT. The reaction mixture is quenched by addition of 1 M citric acid. The layers are separated, and the organic phase is washed with water and brine, dried and concentrated in vacuo. The residue is purified by flash chromatography to give the title compound [HPLC Rt_{B}=4.88 min; ESIMS [M-tBu-H]⁺=454; ¹H-NMR (360 MHz, CDCl₃) 8.16 (d, 2H), 7.36 (d, 2H), 7.25-7.10 (m, 4H), 4.54 (m, 1H), 3.90 (m, 1H), 3.70 (t, 1H), 3.48-3.45 (m, 1H), 3.05-2.80 (m, 4H), 1.45 (s, 9H), 1.38-1.20 (m, 9H).

### c) N—[(S)-1-{(R)-3-[1-(3-Isopropyl-phenyl)-cyclopropyl]-2-oxo-oxazolidin-5-yl}-2-(4-nitro-phenyl)-ethyl]-acetamide

[(S)-1-{(R)-3-[1-(3-Isopropyl-phenyl)-cyclopropyl]-2-oxo-oxazolidin-5-yl}-2-(4-nitro-phenyl)-ethyl]-carbamic acid tert-butyl ester (241.8 mg, 0.5 mmol) is dissolved in DCM (1 ml). The solution is cooled to 0° C. After the addition of TFA (0.2 ml), the mixture is stirred for 30 min at 0° C. and then, after the addition of further TFA (0.4 ml), for 3 h at RT. Toluene (2 ml) is added, and all volatiles are removed in vacuo. The crude mixture is dissolved in pyridine (2 ml), Ac₂O (53 μl, 0.55 mmol) is added, and the mixture is stirred for 5 min. The crude product is used without further purification [HPLC Rt_{B}=4.49 min; ESIMS [M−H]⁺=422].

### d) N—[(S)-2-(4-Amino-phenyl)-1-{(R)-3-[1-(3-isopropyl-phenyl)-cyclopropyl]-2-oxo-oxazolidin-5-yl}-ethyl]-acetamide

N—[(S)-1-{(R)-3-[1-(3-Isopropyl-phenyl)-cyclopropyl]-2-oxo-oxazolidin-5-yl}-2-(4-nitro-phenyl)-ethyl]-acetamide (850 mg, 1.88 mmol) is dissolved in MeOH (20 ml). After cooling to 0° C., NiCl₂×6 H₂O (447 mg, 1.88 mmol) is added in one portion, followed by the addition of NaBH₄ (284 mg, 7.53 mmol). The reaction mixture is quenched by the addition of water, and the volatiles are removed in vacuo. The residue is taken up in EtOAc, and the mixture is filtered through a plug of Celite. The filtrate is washed with saturated NaHCO₃ solution and brine. The organic phase is dried (MgSO₄), filtered and concentrated in vacuo. The product is used without further purification [HPLC Rt_{B}=3.82 min; ESIMS [M−H]⁺=422].

### e) N—[(S)-2-{4-[6-(4-Fluoro-phenyl)-pyrimidin-4-ylamino]-phenyl}-1-{(R)-3-[1-(3-isopropyl-phenyl)-cyclopropyl]-2-oxo-oxazolidin-5-yl}-ethyl]-acetamide

To N—[(S)-2-(4-Amino-phenyl)-1-{(R)-3-[1-(3-isopropyl-phenyl)-cyclopropyl]-2-oxo-oxazolidin-5-yl}-ethyl]-acetamide (100 mg, 0.237 mmol) are added 4-chloro-6-(4-fluoro-phenyl)-pyrimidine (54 mg, 0.26 mmol) and iPrOH (2 ml). To this suspension is added 1 N HCl (0.71 ml, 0.711 mmol), and the reaction mixture is stirred at 150° C. in a microwave for 0.5 h. The mixture is concentrated in vacuo and purified by preparative HPLC (SunFire column 150×19 mm, 5-90% ACN in water+0.1% TFA gradient). The desired fraction is neutralized with saturated aqueous NaHCO₃ solution, and the organic solvents are removed in vacuo. The aqueous phase is extracted with EtOAc, and the combined organic phases are dried with MgSO₄ and concentrated to give the desired product [HPLC Rt_{B}=4.12 min; ESIMS [M−H]⁺=594].

### f) N-{(1S,2R)-1-{4-[6-(4-Fluoro-phenyl)-pyrimidin-4-ylamino]-benzyl}-2-hydroxy-3-[1-(3-isopropyl-phenyl)-cyclopropylamino]-propyl}-acetamide

N—[(S)-2-{-4-[6-(4-Fluoro-phenyl)-pyrimidin-4-ylamino]-phenyl}-1-{(R)-3-[1-(3-isopropyl-phenyl)-cyclopropyl]-2-oxo-oxazolidin-5-yl}-ethyl]-acetamide (55 mg, 0.096 mmol) is dissolved in dry THF (1 ml). KOTMS (37 mg, 0.288 mmol) is added in one portion, and the mixture is stirred at 150° C. for 10 min in a microwave. The mixture is then quenched with 1 N HCl and concentrated in vacuo. The crude product is purified by preparative HPLC (SunFire column 150×19 mm, 5-90% ACN in water+0.1% TFA gradient). The desired fraction is neutralized with saturated aqueous NaHCO₃ solution, and the organic solvents are removed in vacuo. The aqueous phase is extracted with EtOAc, and the combined organic phases are dried with MgSO₄ and concentrated to give the title compound [HPLC Rt_{B}=3.71 min; ESIMS [M−H]⁺=568].

### Example 5

### N-{(1S,2R)-3-[1-(3-tert-Butyl-phenyl)-cyclohexylamino]-1-{4-[6-(4-fluoro-phenyl)-pyrimidin-4-ylamino]-benzyl}-2-hydroxy-propyl}-acetamide hydrochloride

### a) [(1S,2R)-3-[1-(3-tert-Butyl-phenyl)-cyclohexylamino]-2-hydroxy-1-(4-nitro-benzyl)-propyl]-carbamic acid tert-butyl ester

A suspension of [(S)-2-(4-nitro-phenyl)-1-(S)-oxiranyl-ethyl]-carbamic acid tert-butyl ester (0.80 g, 2.54 mmol), LiCl (0.145 g, 3.31 mmol) and 1-(3-tert-butyl-phenyl)-cyclohexylamine (0.90 g, 3.81 mmol) in iPrOH (20 ml) is heated to 50-60° C. for 24 h. The mixture is then diluted with EtOAc (50 ml) and extracted 3× with cold 0.5 N HCl. The organic phase is basified with saturated NaHCO₃ solution, washed with brine, dried over MgSO₄, filtered and evaporated. The residue is purified by flash chromatography (hexane/EtOAc 4:1 to 1:2) to give the product as a yellow oil [TLC (CH₂Cl₂/MeOH 19:1) R_{f}=0.35; HPLC Rt_{c}=2.17 min; ESIMS [M+H]⁺=540; ¹H-NMR (400 MHz, CDCl₃) 8.02 (d, 2H), 7.4 (1H, s), 7.21 (d, 2H), 7.2-7.1 (m, 3H), 4.60 (d, 1H), 3.74 (m, 1H), 3.19 (m, 1H), 2.95 (dd, 1H), 2.68 (dd, 1H), 2.74 (dd, 1H), 2.4 (bs, 1H), 2.24 (m, 2H), 1.9-1.4 (m, 10H), 1.25 (s, 18H)].

### b) {(1S,2R)-1-(4-Amino-benzyl)-3-[1-(3-tert-butyl-phenyl)-cyclohexylamino]-2-hydroxy-propyl}-carbamic acid tert-butyl ester

To a solution of [(1S,2R)-3-[1-(3-tert-butyl-phenyl)-cyclohexylamino]-2-hydroxy-1-(4-nitro-benzyl)-propyl]-carbamic acid tert-butyl ester (0.33 g, 0.616 mmol) in MeOH (5 ml) is added NiCl₂×6 H₂O (0.107 g, 0.616 mmol). To the green mixture is added at 0-5° C. NaBH₄ (0.097 g, 2.46 mmol) in portions within 10-15 min. After stirring for 1 h at 25° C., the reaction is stopped by the slow addition of H₂O (1 ml). The solvents are evaporated, the residue is taken up in EtOAc (30 ml), and the mixture is filtered over Celite. The filtrate is washed with saturated NaHCO₃ solution and brine, dried over MgSO₄ and evaporated. The residue is purified by flash-chromatography (CH₂Cl₂/MeOH 10:1 to 1:10) to give the title compound as a light yellow foam [TLC (CH₂Cl₂/MeOH 10:1) R_{f}=0.50; HPLC Rt_{c}=1.71 min; ESIMS [M+H]⁺=510; ¹H-NMR (400 MHz, CDCl₃) 7.40 (1H, s), 7.25-7.10 (m, 3H), 6.86 (d, 2H), 6.54 (d, 2H), 4.62 (d, 1H), 3.64 (m, 1H), 3.48 (bs, 2H), 3.18 (m, 1H), 2.63 (m, 1H), 2.26 (m, 1H), 2.17 (m, 1H), 1.9-1.2 (m, 28H).

### c) (2R,3S)-3-Amino-1-[1-(3-tert-butyl-phenyl)-cyclohexylamino]-4-{4-[6-(4-fluoro-phenyl)-pyrimidin-4-ylamino]-phenyl}-butan-2-ol hydrochloride

To a solution of {(1S,2R)-1-(4-amino-benzyl)-3-[1-(3-tert-butyl-phenyl)-cyclohexylamino]-2-hydroxy-propyl}-carbamic acid tert-butyl ester (0.20 g, 0.385 mmol) in iPrOH (3 ml) are added 4-chloro-6-(4-fluoro-phenyl)-pyrimidine (0.101 g, 0.462 mmol) and 5 N HCl in iPrOH (0.23 ml). The mixture is heated in a microwave for 0.5 h at 130° C. The solvents are removed, and the dried light yellow residue is used without further purification in the next reaction step [TLC (CH₂Cl₂/MeOH/AcOH/H₂O 180:20:2:1) R_{f}=0.09; HPLC Rt_{c}=1.62 min; ESIMS [M+H]⁺=582].

### d) N-{(1S,2R)-3-[1-(3-tert-Butyl-phenyl)-cyclohexylamino]-1-{4-[6-(4-fluoro-phenyl)-pyrimidin-4-ylamino]-benzyl}-2-hydroxy-propyl}-acetamide hydrochloride

To a solution of (2R,3S)-3-amino-1-[1-(3-tert-butyl-phenyl)-cyclohexylamino]-4-{4-[6-(4-fluoro-phenyl)-pyrimidin-4-ylamino]-phenyl}-butan-2-ol hydrochloride (0.13 g, 0.183 mmol) in DCM (2 ml) is added NEt₃ (0.105 ml, 0.75 mmol) at 0-5° C. To this mixture a 0.1 M solution of Ac₂O (1.9 ml, 0.19 mmol) is added within 15 min. After stirring for 20 min at 0-5° C., the mixture is diluted with CHCl₃ and washed with 5% aqueous K₂CO₃ solution and water. The organic layer is dried over MgSO₄, filtered and evaporated. The residue is purified by flash-chromatography using deactivated silica gel (CH₂Cl₂/MeOH 95:5 to 90:10 containing 0.5% of 2 M NH₃ in EtOH) to give the title compound as a light yellow solid [TLC (CH₂Cl₂/MeOH 19:1+0.5% of 2 M NH₃ in EtOH) R_{f}=0.07; HPLC Rt_{c}=1.72 min; ESIMS [M+H]⁺=624; ¹H-NMR (400 MHz, CD₃OD) 8.8 (s, 1H), 7.9-7.2 (m, 13H), 3.89 (m, 1H), 3.62 (m, 1H), 3.48 (m, 1H), 3.21 (m, 1H), 2.8-2.6 (m, 2H), 2.57 (m, 1H), 2.1-1.2 (m, 19H)].

### Example 6

### N-{(1S,2R)-3-(3-tert-Butyl-benzylamino)-1-{4-[6-(4-fluoro-phenyl)-pyrimidin-4-ylamino]-benzyl}-2-hydroxy-propyl}-acetamide hydrochloride

The title compound is prepared in a manner analogous to that described in Example 5 starting from [(S)-2-(4-nitro-phenyl)-1-(S)-oxiranyl-ethyl]-carbamic acid tert-butyl ester and 3-tert-butyl-benzylamine [TLC (CH₂Cl₂/MeOH 10:1+0.5% of 2 M NH₃ in EtOH) R_{f}=0.23; HPLC Rt_{c}=1.53 min; ESIMS [M+H]⁺=556; ¹H-NMR (600 MHz, DMSO-d₆) 8.67 (s, 1H), 8.07 (m, 2H), 7.73 (d, 1H), 7.56 (d, 2H), 7.4-7.1 (m, 9H), 3.86 (m, 1H), 3.71 (s, 1H), 3.49 (m, 1H), 2.92 (dd, 1H), 2.63 (dd, 1H), 2.53 (m, 2H), 1.70 (s, 3H), 1.24 (s, 9H)].

### Example 7

### N-{(1S,2R)-1-[4-(Biphenyl-3-ylamino)-benzyl]-3-[1-(3-tert-butyl-phenyl)-cyclopropylamino]-2-hydroxy-propyl}-acetamide

### a) [(1S,2R)-3-[1-(3-tert-butyl-phenyl)-cyclopropylamino]-2-hydroxy-1-(4-nitro-benzyl)-propyl]-carbamic acid tert-butyl ester

A suspension of [(S)-2-(4-nitro-phenyl)-1-(S)-oxiranyl-ethyl]-carbamic acid tert-butyl ester (0.492 g, 1.58 mmol) and 1-(3-tert-butyl-phenyl)-cyclopropylamine (0.45 g, 2.37 mmol) in iPrOH (1 ml) is heated to 50-60° C. for 16 h and then to 75° C. for 2 h. The mixture is diluted with EtOAc (50 ml) and extracted 3× with cold 0.5 N HCl. The organic phase is basified with saturated NaHCO₃ solution, washed with brine, dried over MgSO₄, filtered and evaporated. The residue is purified by flash-chromatography (hexane/EtOAc 2:1 to 1:2) to give the title compound as a yellow solid [TLC (hexane/EtOAc 1:1) R_{f}=0.15; HPLC Rt_{c}=2.02 min; ESIMS [M+H]⁺=498; ¹H-NMR (400 MHz, CDCl₃) 8.14 (d, 2H), 7.3-7.1 (m, 4H), 7.16 (d, 2H), 4.54 (d, 1H), 3.78 (m, 1H), 3.36 (m, 1H), 3.14 (dd, 1H), 2.83 (dd, 1H), 2.74 (dd, 1H), 2.63 (dd, 1H), 2.4 (bs, 1H), 1.35 (s, 9H), 1.27 (s, 9H), 0.96 (m, 4H)].

### b) [(S)-1-{(R)-3-[1-(3-tert-butyl-phenyl)-cyclopropyl]-2-oxo-oxazolidin-5-yl}-2-(4-nitro-phenyl)-ethyl]-carbamic acid tert-butyl ester

To a solution of [(1S,2R)-3-[1-(3-tert-butyl-phenyl)-cyclopropylamino]-2-hydroxy-1-(4-nitro-benzyl)-propyl]-carbamic acid tert-butyl ester (0.7 g, 1.39 mmol) in DCE (20 ml) are added carbonyl-diimidazole (0.69 g, 4.18 mmol), DIPEA (0.29 ml, 5.57 mmol) and DMAP (0.009 g, 0.07 mmol). The mixture is heated to reflux for 1 h, then cooled to RT, diluted with DCM and washed with 5% aqueous K₂CO₃ solution, water, cold 0.5 N HCl and water. The organic layer is dried over MgSO₄, filtered and evaporated to give the title compound as a colourless foam used as such in the next reaction step [TLC (hexane/EtOAc 3:1) R_{f}=0.47; HPLC Rt_{c}=2.46 min; ESIMS [M+H+NH₃]⁺=541; ¹H-NMR (400 MHz, CDCl₃) 8.06 (d, 2H), 7.3-7.0 (m, 6H), 4.39 (d, 1H), 4.32 (m, 1H), 3.83 (m, 1H), 3.58 (dd, 1H), 3.34 (dd, 1H), 2.91 (dd, 1H), 2.75 (dd, 1H), 1.35-1.20 (m, 22H)].

### c) [(S)-2-(4-Amino-phenyl)-1-{(R)-3-[1-(3-tert-butyl-phenyl)-cyclopropyl]-2-oxo-oxazolidin-5-yl}-ethyl]-carbamic acid tert-butyl ester

To a solution of [(S)-1-{(R)-3-[1-(3-tert-butyl-phenyl)-cyclopropyl]-2-oxo-oxazolidin-5-yl}-2-(4-nitro-phenyl)-ethyl]-carbamic acid tert-butyl ester (0.65 g, 1.23 mmol) in MeOH (20 ml) is added NiCl₂×6 H₂O (0.212 g, 1.23 mmol). To the green mixture is added in portions NaBH₄ (0.195 g, 4.9 mmol) at 0-5° C. within 10-15 min. After stirring for 20 min at 0-5° C., the reaction is stopped by the slow addition of H₂O (2 ml). The solvents are evaporated, and the residue is taken up in EtOAc (60 ml). The mixture is filtered over Celite. The filtrate is washed with saturated NaHCO₃ solution and brine, dried over MgSO₄, and evaporated. The residue is purified by MPLC (hexane/EtOAc 10:1 to EtOAc) to give the title compound as a light yellow oil [TLC (hexane/EtOAc 1:1) R_{f}=0.24; HPLC Rt_{c}=1.87 min; ESIMS [M+H+NH₃]⁺=511; ¹H-NMR (400 MHz, CDCl₃) 7.35-7.10 (m, 4H), 6.96 (d, 2H), 6.61 (d, 2H), 4.42 (d, 1H), 4.32 (m, 1H), 3.84 (m, 1H), 3.6 (bs, 2H), 3.56 (dd, 1H), 3.40 (dd, 1H), 2.76 (m, 2H), 1.45-1.10 (m, 22H)].

### d) [(S)-2-[4-(Biphenyl-3-ylamino)-phenyl]-1-{(R)-3-[1-(3-tert-butyl-phenyl)-cyclopropyl]-2-oxo-oxazolidin-5-yl}-ethyl]-carbamic acid tert-butyl ester

To a solution of [(S)-2-(4-amino-phenyl)-1-{(R)-3-[1-(3-tert-butyl-phenyl)-cyclopropyl]-2-oxo-oxazolidin-5-yl}-ethyl]-carbamic acid tert-butyl ester (0.2 g, 0.4 mmol) in anhydrous dioxane (10 ml) are added under argon 3-bromobiphenyl (0.145 g, 0.6 mmol), sodium tert-butylate (0.06 g, 0.6 mmol), Pd₂(dba)₃ (0.019 g, 0.02 mmol) and 2-dicyclohexylphosphino-2′,6′-dimethoxy-biphenyl (0.022 g, 0.05 mmol). The mixture is heated to reflux for 2 h, then cooled to RT and diluted with EtOAc. The organic phase is washed with saturated NaHCO₃ solution and brine, dried over MgSO₄ and evaporated. The residue is purified by MPLC (hexane/EtOAc 10:1 to EtOAc) to give the title compound as a light yellow oil [TLC (hexane/EtOAc 1:1) R_{f}=0.49; HPLC Rt_{c}=2.83 min; ESIMS [M+H+NH₃]⁺=663; ¹H-NMR (400 MHz, CDCl₃) 7.56 (d, 2H), 7.43 (t, 2H), 7.4-7.0 (m, 9H), 5.8 (s, 1H), 4.46 (d, 1H), 4.35 (m, 1H), 3.88 (m, 1H), 3.59 (dd, 1H), 3.42 (dd, 1H), 2.81 (m, 2H), 1.45-1.20 (m, 22H)].

### e) (2R,3S)-3-Amino-4-[4-(biphenyl-3-ylamino)-phenyl]-1-[1-(3-tert-butyl-phenyl)-cyclopropylamino]-butan-2-ol hydrochloride

To a solution of [(S)-2-[4-(biphenyl-3-ylamino)-phenyl]-1-{(R)-3-[1-(3-tert-butyl-phenyl)-cyclopropyl)-2-oxo-oxazolidin-5-yl}-ethyl]-carbamic acid tert-butyl ester (0.095 g, 0.146 mmol) in anhydrous THF (6 ml) is added under argon KOTMS (0.062 g, 0.438 mmol). The mixture is heated to reflux for 8 h, after cooling to RT neutralized with 1 N HCl in Et₂O and evaporated to dryness. The residue is taken up in CHCl₃, the mixture is evaporated, the residue is taken up again in CHCl₃, and the mixture is evaporated and dried to give the crude title compound used as such in the next reaction step [TLC (CH₂Cl₂/MeOH/AcOH/H₂O 180:20:2:1) R_{f}=0.16; HPLC Rt_{c}=1.98 min; ESIMS [M+H]⁺=520].

### f) N-{(1S,2R)-1-[4-(Biphenyl-3-ylamino)-benzyl]-3-[1-(3-tert-butyl-phenyl)-cyclopropylamino]-2-hydroxy-propyl}-acetamide

To a solution of (2R,3S)-3-amino-4-[4-(biphenyl-3-ylamino)-phenyl]-1-[1-(3-tert-butyl-phenyl)-cyclopropylamino]-butan-2-ol hydrochloride (0.056 mg, 0.108 mmol) in DCM (6 ml) is added NEt₃ (0.06 ml, 0.43 mmol) at 0-5° C. To this mixture a 0.1 M solution of Ac₂O (1.2 ml, 0.12 mmol) is added within 2-3 min. The mixture is stirred for 15 min at 0-5° C., diluted with CHCl₃ and washed with 5% aqueous K₂CO₃ solution and water. The organic layer is dried over MgSO₄, filtered and evaporated. The residue is purified by flash-chromatography on silica (CH₂Cl₂/MeOH/Et₂O 95:5:50 to 90:10:0) to give the title compound as light yellow solid [TLC (CH₂Cl₂/MeOH 10:1) R_{f}=0.38; HPLC Rt_{c}=2.19 min; ESIMS [M+H]⁺=562; ¹H-NMR (400 MHz, CDCl₃) 7.56 (d, 2H), 7.42 (t, 2H), 7.36-7.00 (m, 13H), 5.76 (s, 1H), 5.6 (d, 1H), 4.09 (m, 1H), 3.42 (m, 1H), 3.3 (bs, 1H), 2.86 (dd, 1H), 2.82 (dd, 1H), 2.65 (d, 2H), 2.2 (bs, 1H), 1.83 (s, 3H), 1.33 (s, 9H), 1.40-1.15 (m, 4H)].

### Example 8

### N-{(1S,2R)-1-[4-(Biphenyl-3-ylamino)-benzyl]-2-hydroxy-3-[1-(3-isopropyl-phenyl)-cyclopropylamino]-propyl}-acetamide

The title compound is prepared in a manner analogous to that described in Example 4 [HPLC Rt_{B}=4.25 min; ESIMS [M−H]⁺=548].

### Example 9

### N-{(1S,2R)-2-Hydroxy-3-[1-(3-isopropyl-phenyl)-cyclopropylamino]-1-[4-(4-phenyl-pyridin-2-ylamino)-benzyl]-propyl}-acetamide

### a) N-{(S)-1-{(R)-3-[1-(3-Isopropyl-phenyl)-cyclopropyl]-2-oxo-oxazolidin-5-yl}-2-[4-(4-phenyl-pyridin-2-ylamino)-phenyl]-ethyl}-acetamide

N—[(S)-2-(4-Amino-phenyl)-1-{(R)-3-[1-(3-isopropyl-phenyl)-cyclopropyl]-2-oxo-oxazolidin-5-yl}-ethyl]-acetamide (100 mg, 0.237 mmol), 2-chloro-4-phenyl-pyridine (63 mg, 0.33 mmol), Pd₂(dba)₃ (11 mg, 0.012 mmol), 2-dicyclohexylphosphino-2′-(N,N-dimethylamino)-biphenyl (9.3 mg, 0.24 mmol) and sodium tert-butoxide (35 mg, 0.36 mmol) are dissolved in dry dioxane (2.0 ml). The reaction mixture is heated to 100° C. for 3 h, then cooled to RT and filtered over a bed of Celite. The filter cake is washed with EtOAc, and the combined filtrates are washed with brine, dried over Na₂SO₄, filtered and concentrated. The residue is purified on silica to give the title compound [HPLC Rt_{B}=4.08 min; ESIMS [M−H]⁺=575].

### b) N-{(1S,2R)-2-Hydroxy-3-[1-(3-isopropyl-phenyl)-cyclopropylamino]-1-[4-(4-phenyl-pyridin-2-ylamino)-benzyl]-propyl}-acetamide

The title compound is prepared in a manner analogous to that described in Example 4f [HPLC Rt_{B}=3.72 min; ESIMS [M−H]⁺=549].

### Example 10

### N-{(1S,2R)-3-[1-(3-tert-Butyl-phenyl)-cyclopropylamino]-1-{-4-[5-(4-fluoro-phenyl)-isoxazol-3-ylamino]benzyl}-2-hydroxy-propyl}-acetamide hydrochloride

### a) [(S)-1-{(R)-3-[1-(3-tert-Butyl-phenyl)-cyclopropyl]-2-oxo-oxazolidin-5-yl}-2-{-4-[3-(4-fluoro-phenyl)-3-oxo-propionylamino]-phenyl}-ethyl]-carbamic acid tert-butyl ester

A solution of [(S)-2-(4-amino-phenyl)-1-{(R)-3-[1-(3-tert-butyl-phenyl)-cyclopropyl]-2-oxo-oxazolidin-5-yl}-ethyl]-carbamic acid tert-butyl ester (0.90 g, 1.79 mmol) and 3-(4-fluoro-phenyl)-3-oxo-propionic kid methyl ester (0.37 g, 1.72 mmol) in toluene/DMF 3:1 (20 ml) is heated to 130° C. for 4 h. The reaction mixture is poured onto cold NaH₂PO₄ solution. The mixture is extracted with EtOAc, and the combined extracts are washed with brine, dried over MgSO₄ and evaporated. The residue is purified by flash-chromatography on silica gel (toluene/EtOAc 2:1) to give the title compound as a yellow solid [TLC (toluene/EtOAc 1:1) R_{f}=0.40; HPLC Rt_{c}=2.42 min; ESIMS [M+H+NH₃]⁺=675; ¹H-NMR (400 MHz, CDCl₃) 9.16 (s, 1H), 8.08 (m, 2H), 7.50 (d, 2H), 7.4-7.1 (m, 8H), 4.42 (m, 1H), 4.33 (m, 1H), 4.06 (s, 1H), 3.87 (m, 1H), 3.65 (m, 1H), 3.57 (m, 1H), 3.41 (m, 1H), 2.82 (m, 2H), 1.36 (s, 9H), 1.31 (s, 9H), 1.14 (d, 4H)].

### b) [(S)-1-{(R)-3-[1-(3-tert-Butyl-phenyl)-cyclopropyl]-2-oxo-oxazolidin-5-yl}-2-{4-[(Z)-3-(4-fluoro-phenyl)-1-mercapto-3-oxo-propenylamino]-phenyl}-ethyl]-carbamic acid tert-butyl ester

A mixture of [(S)-1-{(R)-3-[1-(3-tert-butyl-phenyl)-cyclopropyl]-2-oxo-oxazolidin-5-yl}-2-{4-[3-(4-fluoro-phenyl)-3-oxo-propionylamino]-phenyl}-ethyl]-carbamic acid tert-butyl ester (1.05 g, 1.59 mmol) and Lawesson reagent (0.72 g, 1.75 mmol) is stirred in DCE (30 ml) for 36 h at 25° C. and then diluted with aqueous NaH₂PO₄ solution. The mixture is extracted with EtOAc, and the combined extracts are washed with brine, dried over MgSO₄ and evaporated. The residue is purified by flash-chromatography on silica gel (toluene/EtOAc 2:1) to give the title compound as a yellow resin [TLC (toluene/EtOAc 1:1) R_{f}=0.58; ESIMS [M+H]⁺=674].

### c) [(S)-1-{(R)-3-[1-(3-tert-Butyl-phenyl)-cyclopropyl]-2-oxo-oxazolidin-5-yl}-2-{4-[(Z)-3-(4-fluoro-phenyl)-1-methylsulfanyl-3-oxo-propenylamino]-phenyl}-ethyl]-carbamic acid tert-butyl ester

To a solution of [(S)-1-{(R)-3-[1-(3-tert-butyl-phenyl)-cyclopropyl]-2-oxo-oxazolidin-5-yl}-2-{4-[(Z)-3-(4-fluoro-phenyl)-1-mercapto-3-oxo-propenylamino]-phenyl}-ethyl]-carbamic acid tert-butyl ester (0.70 g, 1.03 mmol) in anhydrous THF (10 ml) is added 60% NaH in oil (0.040 g, 1.03 mmol) in portions at 0-5° C. After stirring for 10 min, methyl iodide (0.15 g, 1.03 mmol) is added, and the mixture is stirred for 1 h at 25° C. and then poured onto cold NH₄Cl solution. The mixture is extracted with EtOAc, and the combined extracts are washed with brine, dried over MgSO₄ and evaporated. The residue is purified by MPLC on silica gel (hexane/EtOAc 10:1 to 5:95) to give the title compound as a yellow solid [TLC (hexane/EtOAc 1:1) R_{f}=0.44; HPLC Rt_{c}=2.71 min; ESIMS [M+H]⁺=688; ¹H-NMR (400 MHz, CDCl₃) 13.4 (s, 1H), 7.92 (m, 2H), 7.4-7.1 (m, 10H), 5.81 (s, 1H), 4.44 (m, 1H), 4.38 (m, 1H), 3.92 (m, 1H), 3.62 (dd, 1H), 3.42 (dd, 1H), 2.92 (m, 1H), 2.81 (m, 1H), 2.42 (s, 3H), 1.37 (s, 9H), 1.3-1.1 (s, 13H)].

### d) [(S)-1-{(R)-3-[1-(3-tert-Butyl-phenyl)-cyclopropyl]-2-oxo-oxazolidin-5-yl}-2-{4-[5-(4-fluoro-phenyl)-isoxazol-3-ylamino]-phenyl}-ethyl]-carbamic acid tert-butyl ester

A mixture of [(S)-1-{(R)-3-[1-(3-tert-butyl-phenyl)-cyclopropyl]-2-oxo-oxazolidin-5-yl}-2-{4-[(Z)-3-(4-fluoro-phenyl)-1-methylsulfanyl-3-oxo-propenylamino]-phenyl}-ethyl]-carbamic acid tert-butyl ester (0.266 g, 0.387 mmol), hydroxylamine hydrochloride (0.055 g, 0.774 mmol) and Na₂CO₃ (0.092 g, 0.85 mmol) in EtOH (5 ml) is heated to 70° C. for 2 h. The mixture is cooled to RT and diluted with aqueous NaH₂PO₄ solution. The mixture is extracted with EtOAc, and the combined extracts are washed with brine, dried over MgSO₄ and evaporated. The residue is purified by MPLC on silica gel (hexane/EtOAc 10:1 to 5:95) to give the title compound as a light yellow solid [TLC (toluene/EtOAc 1:1) R_{f}=0.35; HPLC Rt_{c}=2.62 min; ESIMS [M+H]⁺=655].

### e) (2R,3S)-3-Amino-1-[1-(3-tert-butyl-phenyl)-cyclopropylamino]-4-{4-[5-(4-fluoro-phenyl)-isoxazol-3-ylamino]-phenyl}-butan-2-ol

A solution of [(S)-1-{(R)-3-[1-(3-tert-butyl-phenyl)-cyclopropyl]-2-oxo-oxazolidin-5-yl}-2-{4-[5-(4-fluoro-phenyl)-isoxazol-3-ylamino]-phenyl}-ethyl]-carbamic acid tert-butyl ester (0.24 g, 0.367 mmol) and KOTMS (0.246 g, 1.725 mmol) in THF (3 ml) is heated to reflux for 3 h. The cold reaction mixture is diluted with aqueous NaH₂PO₄ solution and extracted with EtOAc. The combined extracts are washed with brine, dried over MgSO₄ and evaporated to give the title compound as a beige solid used as such in the next reaction step [HPLC Rt_{c}=1.85 min; ESIMS [M+H]⁺=529].

### f) N-{(1S,2R)-3-[1-(3-tert-Butyl-phenyl)-cyclopropylamino]-1-{4-[5-(4-fluoro-phenyl)-isoxazol-3-ylamino]-benzyl}-2-hydroxy-propyl}-acetamide hydrochloride

The title compound is prepared in free base form in a manner analogous to that described in Example 7f and then converted into the hydrochloride with 1 N HCl in Et₂O [TLC (EtOAc) R_{f}=0.11; HPLC Rt_{c}=2.00 min; ESIMS [M+H]⁺=571; ¹H-NMR (400 MHz, CD₃OD) 7.83 (m, 2H), 7.65 (s, 1H), 7.5-7.1 (m, 9H), 6.40 (s, 1H), 3.84 (m, 1H), 3.73 (m, 1H), 3.11 (dd, 1H), 3.02 (dd, 1H), 2.94 (dd, 1H), 2.54 (dd, 1H), 1.80 (s, 3H), 1.5-1.1 (m, 13H)].

### Example 11

### N-{(1S,2R)-3-[1-(3-tert-Butyl-phenyl)-cyclopropylamino]-1-{4-[5-(4-fluoro-phenyl)-1-methyl-1H-pyrazol-3-ylamino]-benzyl}-2-hydroxy-propyl}-acetamide hydrochloride

### a) [(S)-1-{(R)-3-[1-(3-tert-Butyl-phenyl)-cyclopropyl]-2-oxo-oxazolidin-5-yl}-2-{4-[5-(4-fluoro-phenyl)-1-methyl-1H-pyrazol-3-ylamino]-phenyl}-ethyl]-carbamic acid tert-butyl ester

The title compound is prepared in a manner analogous to that described in Example 10d, starting from [(S)-1-{(R)-3-[1-(3-tert-butyl-phenyl)-cyclopropyl]-2-oxo-oxazolidin-5-yl}-2-{4-[(Z)-3-(4-fluoro-phenyl)-1-methylsulfanyl-3-oxo-propenylamino]-phenyl}-ethyl]-carbamic acid tert-butyl ester and methyl-hydrazine [TLC (hexane/EtOAc 1:1) R_{f}=0.25; HPLC Rt_{c}=2.46 min; ESIMS [M+H]⁺=667; ¹H-NMR (400 MHz, CDCl₃) 7.73 (m, 2H), 7.72 (d, 2H), 7.4-7.0 (m, 8H), 6.26 (s, 1H), 5.26 (s, 1H), 4.42 (m, 1H), 4.35 (m, 1H), 3.87 (m, 1H), 3.74 (s, 3H), 3.59 (dd, 1H), 3.42 (dd, 1H), 2.9-2.7 (m, 2H), 1.37 (s, 9H), 1.3-1.1 (s, 13H)].

### b) (2R,3S)-3-Amino-1-[1-(3-tert-butyl-phenyl)-cyclopropylamino]-4-{4-[5-(4-fluoro-phenyl)-1-methyl-1H-pyrazol-3-ylamino]-phenyl}-butan-2-ol

The title compound is prepared in a manner analogous to that described in Example 10e [HPLC Rt_{c}=1.74 min; ESIMS [M+H]⁺=542].

### c) N-{(1S,2R)-3-[1-(3-tert-Butyl-phenyl)-cyclopropylamino]-1-{4-[5-(4-fluoro-phenyl)-1-methyl-1H-pyrazol-3-ylamino]-benzyl}-2-hydroxy-propyl}-acetamide hydrochloride

The title compound is prepared in free base form in a manner analogous to that described in Example 7f and then converted into the hydrochloride with 1 N HCl in Et₂O [TLC (EtOAc/MeOH 9:1) R_{f}=0.40; HPLC Rt_{c}=1.87 min; ESIMS [M+H]⁺=584].

### Example 12

### 2,2,2-Trifluoro-N-{(1S,2R)-2-hydroxy-3-[1-(3-isopropyl-phenyl)-cyclopropylamino]-1-[4-(6-phenyl-pyrimidin-4-ylamino)-benzyl]-propyl}-acetamide

### a) (R)-5-{(S)-1-Amino-2-[4-(6-phenyl-pyrimidin-4-ylamino)-phenyl]-ethyl}-3-[1-(3-isopropyl-phenyl)-cyclopropyl]-oxazolidin-2-one

[(S)-2-(4-Amino-phenyl)-1-{(R)-3-[1-(3-isopropyl-phenyl)-cyclopropyl]-2-oxo-oxazolidin-5-yl}-ethyl]-carbamic acid tert-butyl ester (335.7 mg, 0.70 mmol) and 4-chloro-6-phenyl-pyrimidine (140.1 mg, 0.73 mmol) are dissolved in iPrOH (3.0 ml). To this mixture is added 1 N HCl (2.1 ml, 2.1 mmol). The reaction mixture is heated in a microwave to 150° C. for 10 min. The volatiles are removed by evaporation, and the residue is partitioned between EtOAc and saturated aqueous NaHCO₃ solution. The aqueous phase is extracted with EtOAc, and the combined organic layers are washed with brine, dried over MgSO₄, filtered and concentrated in vacuo. The residue is purified on silica to give the title compound [HPLC Rt_{B}=3.77 min; ESIMS [M−H]⁺=534].

### b) (2R,3S)-3-Amino-1-[1-(3-isopropyl-phenyl)-cyclopropylamino]-4-[4-(6-phenyl-pyrimidin-4-ylamino)-phenyl]-butan-2-ol hydrochloride

(R)-5-{(S)-1-Amino-2-[4-(6-phenyl-pyrimidin-4-ylamino)-phenyl]-ethyl}-3-[1-(3-isopropyl-phenyl)-cyclopropyl]-oxazolidin-2-one (43 mg, 0.08 mmol) is dissolved in THF (1.0 ml). KOTMS is added in one portion, and the mixture is stirred at 150° C. in a microwave for 10 min. The reaction mixture is quenched by the addition of 6 N HCl in Et₂O and concentrated. The crude product is reacted further without additional purification [HPLC Rt_{B}=3.49 min; ESIMS [M−H]⁺=508].

### c) 2,2,2-Trifluoro-N-{(1S,2R)-2-hydroxy-3-[1-(3-isopropyl-phenyl)-cyclopropylamino]-1-[4-(6-phenyl-pyrimidin-4-ylamino)-benzyl]-propyl}-acetamide

(2R,3S)-3-Amino-1-[1-(3-isopropyl-phenyl)-cyclopropylamino]-4-[4-(6-phenyl-pyrimidin-4-ylamino)-phenyl]-butan-2-ol (41 mg, 0.08 mmol) is dissolved in dry DCM (2 ml). The mixture is cooled with an ice-bath, and NEt₃ followed by TFAA is added. After stirring for 10 min, the reaction mixture is subjected to basic work-up to give the title compound in pure form after silica gel chromatography [HPLC Rt_{B}=3.80 min; ESIMS [M−H]⁺=604].

### Example 13

### N-{(1S,2R)-2-Hydroxy-3-[1-(3-isopropyl-phenyl)-cyclopropylamino]-1-[4-(6-phenyl-pyrimidin-4-ylamino)-benzyl]-propyl}-2-methoxy-acetamide

### a) N-{(S)-1-{(R)-3-[1-(3-Isopropyl-phenyl)-cyclopropyl]-2-oxo-oxazolidin-5-yl}-2-[4-(6-phenyl-pyrimidin-4-ylamino)-phenyl]-ethyl}-2-methoxy-acetamide

(R)-5-{(S)-1-Amino-2-[4-(6-phenyl-pyrimidin-4-ylamino)-phenyl]-ethyl}-3-[1-(3-isopropyl-phenyl)-cyclopropyl]-oxazolidin-2-one (81 mg, 0.15 mmol) and methoxyacetic acid (24 μl, 0.30 mmol) are dissolved in DCM. NEt₃ (63 μl, 0.45 mmol) and P3P (148 μl, 0.60 mmol) are added, and the mixture is stirred at RT. The reaction mixture is quenched by the addition of saturated aqueous NaHCO₃ solution and worked up. The crude product is purified by silica gel chromatography to give the pure title compound [HPLC Rt_{B}=4.19 min; ESIMS [M−H]⁺=606].

### b) N-{(1S,2R)-2-Hydroxy-3-[1-(3-isopropyl-phenyl)-cyclopropylamino]-1-[4-(6-phenyl-pyrimidin-4-ylamino)-benzyl]-propyl}-2-methoxy-acetamide

The title compound is prepared in a manner analogous to that described in Example 12b [HPLC Rt_{B}=3.77 min; ESIMS [M−H]⁺=580].

### Examples 14 to 16

The compounds listed in Table 1 can be prepared in a manner analogous to that described in Example 12.

TABLE 1

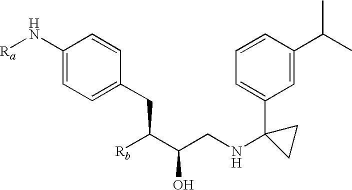

| Example | Rₐ | R_{b} | HPLC | ESIMS |
|---|---|---|---|---|
| 14 | 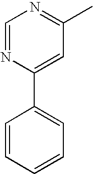 | 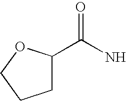 | Rt_{B} = 3.77 min | [M − H]⁺ = 606 |
| 15 | 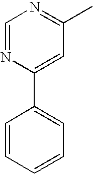 | 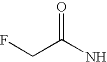 | Rt_{B} = 4.01 min | [M − H]⁺ = 568 |
| 16 | 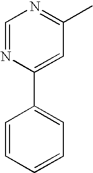 | 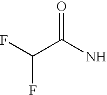 | Rt_{B} = 4.21 min | [M − H]⁺ = 586 |

### Example 17

### 1-{(1S,2R)-2-Hydroxy-3-[1-(3-isopropyl-phenyl)-cyclopropylamino]-1-[4-(6-phenyl-pyrimidin-4-ylamino)-benzyl]propyl}-pyrrolidin-2-one trifluoroacetate

### a) 4-{(1S,2R)-2-Hydroxy-3-[1-(3-isopropyl-phenyl)-cyclopropylamino]-1-[4-(6-phenyl-pyrimidin-4-ylamino)-benzyl]-propylamino}-butyric acid

(R)-6-{(S)-1-Amino-2-[4-(6-phenyl-pyrimidin-4-ylamino)-phenyl]-ethyl}-4-[1-(3-isopropyl-phenyl)-cyclopropyl]-morpholin-3-one (240 mg, 0.5 mmol) is dissolved in MeOH (2.5 ml). Methyl-4-oxobutanoate (88 mg, 0.7 mmol) is added, followed by polymer-supported borohydride (3 mmol/g, 500 mg, 1.5 mmol), and the suspension is stirred. The polymer is filtered off, and the filtrate is concentrated to give product, which is reacted in the next step without further purification. The ester thus produced is dissolved in anhydrous THF (1 ml), KOTMS (19 mg, 0.13 mmol) is added, and the suspension is stirred at 150° C. in a microwave for 10 min. The reaction mixture is acidified with 1 N HCl in Et₂O and concentrated in vacuo. The residue is taken up in CHCl₃, and the mixture is evaporated. This is repeated once. The crude product is purified by preparative HPLC (Sunfire column 19×150 mm; 5 μm; gradient 5-90% H₂O in ACN+0.1% TFA). The desired fraction is neutralized with saturated aqueous sodium bicarbonate, and the organic solvents are removed in vacuo. The aqueous residue is extracted with EtOAc, and the combined organic fractions are dried with MgSO₄ and concentrated to give the title compound [HPLC Rt_{B}=3.60 min; ESIMS [M−H]⁺=595].

### b) 1-{(1S,2R)-2-Hydroxy-3-[1-(3-isopropyl-phenyl)-cyclopropylamino]-1-[4-(6-phenyl-pyrimidin-4-ylamino)-benzyl]-propyl}-pyrrolidin-2-one trifluoroacetate

4-{(1S,2R)-2-Hydroxy-3-[1-(3-isopropyl-phenyl)-cyclopropylamino]-1-[4-(6-phenyl-pyrimidin-4-ylamino)-benzyl]-propylamino}-butyric acid (190 mg, 0.3 mmol) is dissolved in dry DMF (1 ml). BOP-Cl (90 mg, 0.4 mmol) is added, followed by NaHCO₃ (538 mg, 6.4 mmol). The suspension is stirred at RT. After aqueous work-up, the crude material is purified by preparative HPLC (Sunfire column 19×150 mm; 5 μm; gradient 5-90% H₂O in ACN+0.1% TFA) to give the title compound [HPLC Rt_{B}=3.60 min; ESIMS [M−H]⁺=576].

### Example 18

### N-{(1S,2R)-2-Hydroxy-3-[1-(3-isopropyl-phenyl)-cyclopropylamino]-1-[4-(6-methoxy-pyrimidin-4-ylamino)-benzyl]-propyl}-acetamide

The title compound can be prepared in a manner analogous to that described in Example 5 [HPLC Rt_{B}=3.70 min; ESIMS [M−H]⁺=504].

### Example 19

N-{(1S,2R)-1-[4-(2,6-Dimethoxy-pyrimidin-4-ylamino)-benzyl]-2-hydroxy-3-[1-(3-isopropyl-phenyl)-cyclopropylamino]-propyl}-acetamide

The title compound can be prepared in a manner analogous to that described in Example 5 [HPLC Rt_{B}=3.82 min; ESIMS [M−H]⁺=534].

### Example 20

N-{(1S,2R)-3-[1-(3-tert-Butyl-phenyl)-cyclopropylamino]-2-hydroxy-1-[4-(2-isopropyl-6-methyl-pyrimidin-4-ylamino)-benzyl]-propyl}-acetamide

The title compound can be prepared in a manner analogous to that described in Example 5 [HPLC Rt_{c}=1.55 min; ESIMS [M−H⁺=544].

### Example 21

### N-((1S,2R)-3-[1-(3-tert-Butyl-phenyl)-cyclopropylamino]-2-hydroxy-1-{-4-[2-(2-hydroxy-phenyl)-6-methyl-pyrimidin-4-ylamino]-benzyl}-propyl)-acetamide

The title compound can be prepared in a manner analogous to that described in Example 5 [HPLC Rt_{c}=1.64 min; ESIMS [M−H]⁺=594].

### Example 22

### N-{(1S,2R)-3-[1-(3-tert-Butyl-phenyl)-cyclopropylamino]-1-[4-(2,6-dimethyl-pyrimidin-4-ylamino)-benzyl]-2-hydroxy-propyl}-acetamide

The title compound can be prepared in a manner analogous to that described in Example 5 [HPLC Rt_{D}=1.52 min; ESIMS [M−H]⁺=516].

### Example 23

### N-{(1S,2R)-3-[1-(3-tert-Butyl-phenyl)-cyclopropylamino]-1-[4-(2-chloro-6-methyl-pyrimidin-4-ylamino)-benzyl]-2-hydroxy-propyl}-acetamide

The title compound can be prepared in a manner analogous to that described in Example 5 [HPLC Rt_{D}=2.22 min; ESIMS [M−H]⁺=536, 538].

### Example 24

### N-{(1S,2R)-3-[1-(3-tert-Butyl-phenyl)-cyclopropylamino]-1-[4-(2-chloro-pyrimidin-4-ylamino)-benzyl]-2-hydroxy-propyl}-acetamide

The title compound can be prepared in a manner analogous to that described in Example 5 [HPLC Rt_{D}=2.15 min; ESIMS [M−H]⁺=522, 524].

### Example 25

### N-{(1S,2R)-3-[1-(3-tert-Butyl-phenyl)-cyclopropylamino]-1-[4-(2-chloro-6-ethyl-pyrimidin-4-ylamino)-benzyl]-2-hydroxy-propyl}-acetamide

The title compound can be prepared in a manner analogous to that described in Example 5 [HPLC Rt_{B}=3.96 min; ESIMS [M−H]⁺=550, 552].

### Examples 26 to 40

The compounds listed in Table 2 can be prepared in a manner analogous to that described in Example 9.

TABLE 2

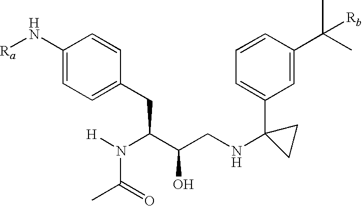

| Example | Rₐ | R_{b} | HPLC | ESIMS |
|---|---|---|---|---|
| 26 | 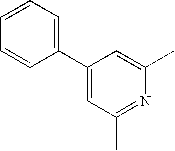 | H | Rt_{B} = 3.68 min | [M − H]⁺ = 563 |
| 27 | 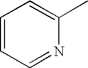 | H | Rt_{B} = 3.45 min | [M − H]⁺ = 473 |
| 28 | 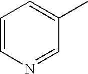 | H | Rt_{B} = 3.43 min | [M − H]⁺ = 473 |
| 29 | 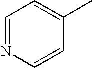 | H | Rt_{B} = 3.43 min | [M − H]⁺ = 473 |
| 30 | 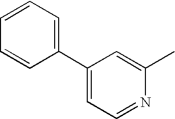 | H | Rt_{B} = 3.66 min | [M − H]⁺ = 549 |
| 31 | 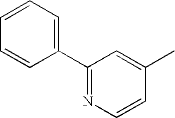 | H | Rt_{B} = 3.64 min | [M − H]⁺ = 549 |
| 32 | 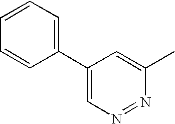 | H | Rt_{B} = 3.70 min | [M − H]⁺ = 550 |
| 33 | 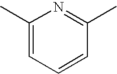 | H | Rt_{B} = 3.50 min | [M − H]⁺ = 487 |
| 34 | 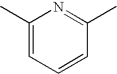 | CH₃ | Rt_{D} = 1.63 min | [M − H]⁺ = 501 |
| 35 | 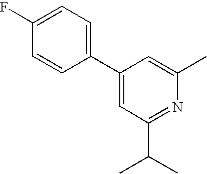 | CH₃ | Rt_{C} = 1.87 min | [M − H]⁺ = 623 |
| 36 | 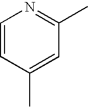 | CH₃ | Rt_{B} = 3.60 min | [M − H]⁺ = 501 |
| 37 | 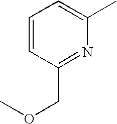 | CH₃ | Rt_{C} = 1.51 min | [M − H]⁺ = 531 |
| 38 | 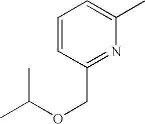 | CH₃ | Rt_{C} = 1.61 min | [M − H]⁺ = 559 |
| 39 | 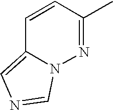 | H | Rt_{B} = 3.47 min | [M − H]⁺ = 513 |
| 40 | 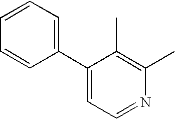 | CH₃ | Rt_{D} = 2.44 min | [M − H]⁺ = 577 |

### Example 41

### N-{(1S,2R)-1-{4-[6-methylpyridin-2-ylamino]-3-pentyl-benzyl}-2-hydroxy-3-[1-(3-tert-butyl-phenyl)-cyclopropylamino]-propyl}-acetamide

### a) ((S)-2-(3-Bromo-4-amino-phenyl)-1-{(R)-3-[1-(3-tert-butyl-phenyl)-cyclopropyl]-2-oxo-oxazolidin-5-yl}-ethyl)-carbamic acid tert-butyl ester

To a solution of ((S)-2-(4-amino-phenyl)-1-{(R)-3-[1-(3-tert-butyl-phenyl)-cyclopropyl]-2-oxo-oxazolidin-5-yl}-ethyl)-carbamic acid tert-butyl ester (1.05 g, 2.12 mmol) in anhydrous DCM (25 ml) is added dropwise 1-butyl-3-methylimidazolium tribromide (852 mg, 2.25 mmol) prepared from 1-butyl-3-methylimidazolium bromide (1 g, 4.56 mmol) and bromine (730 mg, 4.56 mmol) at −10° C. After 10 min the solution is transferred into a separation funnel with Et₂O and washed with thiosulfate-solution, saturated NaHCO₃ solution and brine, dried over Na₂SO₄, filtered and evaporated. The residue is purified by MPLC using (hexane-EtOAc 85:15) to give the product as a white foam [TLC (hexane-EtOAc 1:1) R_{f}=0.48; ESIMS [M+H+NH₃]⁺=592, 590].

### b) ((S)-2-(3-Bromo-4-trifluoroacetamido-phenyl)-1-{(R)-3-[1-(3-tert-butyl-phenyl)-cyclopropyl]-2-oxo-oxazolidin-5-yl}-ethyl)-carbamic acid tert-butyl ester

To a solution of ((S)-2-(3-Bromo-4-amino-phenyl)-1-{(R)-3-[1-(3-tert-butyl-phenyl)-cyclopropyl]-2-oxo-oxazolidin-5-yl}-ethyl)-carbamic acid tert-butyl ester (200 mg, 0.35 mmol) in anhydrous DCE (10 ml) and pyridine (2 ml) is added slowly TFAA (56 μl, 0.4 mmol) at room temperature. After 1 h more TFAA (100 μl, 0.7 mmol) is added. The solution is diluted with EtOAc after 16 h and washed with 5% NaHSO₄ solution, saturated NaHCO₃ solution and brine, dried over Na₂SO₄, filtered and evaporated. The residue is purified by MPLC using (hexane-EtOAc 2:1) to give the product: TLC (hexane-EtOAc 3:1) Rf=0.17; ESIMS [M+H+NH₃]⁺=685/687.

### c) ((S)-2-(3-Pentynyl-4-trifluoroacetamido-phenyl)-1-{(R)-3-[1-(3-tert-butyl-phenyl)-cyclopropyl]-2-oxo-oxazolidin-5-yl}-ethyl)-carbamic acid tert-butyl ester

To a degassed solution of ((S)-2-(3-Bromo-4-trifluoroacetamido-phenyl)-1-{(R)-3-[1-(3-tert-butyl-phenyl)-cyclopropyl]-2-oxo-oxazolidin-5-yl}-ethyl)-carbamic acid tert-butyl ester (189 mg, 0.28 mmol) and tributyl-1-pentynyl stannane (132 mg, 0.37 mmol) in anhydrous toluene (3 ml) is added tetrakis-triphenylphosphino palladium (40 mg, 0.03 mmol) under argon. The solution is filtered through silica (hexane-EtOAc 2:1) and purified by MPLC using (hexane-EtOAc 4:1) to give the product: TLC (hexane-EtOAc 2:1) Rf=0.51; ESIMS [M+H+NH₃]⁺=0.673.

### d) ((S)-2-(3-Pentyl-4-trifluoroacetamido-phenyl)-1-{(R)-3-[1-(3-tert-butyl-phenyl)-cyclopropyl]-2-oxo-oxazolidin-5-yl}-ethyl)-carbamic acid-tert-butyl ester

A solution of ((S)-2-(3-pentynyl-4-trifluoroacetamido-phenyl)-1-{(R)-3-[1-(3-tert-butyl-phenyl)-cyclopropyl]-2-oxo-oxazolidin-5-yl}-ethyl)-carbamic acid tert-butyl ester (165 mg, 0.25 mmol) in EtOAc (150 ml) is hydrogenated over 5% Pd/C at room temperature and 1 mbar. After 15 min the catalyst is filtered off and the solution evaporated to yield the product: TLC (hexane-EtOAc 2:1) Rf=0.41; ESIMS [M+H+NH₃]⁺=0.677.

### e) ((S)-2-(3-Pentyl-4-amino-phenyl)-1-{(R)-3-[1-(3-tert-butyl-phenyl)-cyclopropyl]-2-oxo-oxazolidin-5-yl}-ethyl)-carbamic acid tert-butyl ester

A solution of ((S)-2-(3-pentyl-4-trifluoroacetamido-phenyl)-1-{(R)-3-[1-(3-tert-butyl-phenyl)-cyclopropyl]-2-oxo-oxazolidin-5-yl}-ethyl)-carbamic acid tert-butyl ester (146 mg, 0.22 mmol) in dioxane (3 ml) and water (1 ml) containing 35% NaOH (0.5 ml) is treated in the microwave oven at 100° C. for 3 h. The solution is diluted with EtOAc and washed with water and brine, dried over Na₂SO₄, filtered and evaporated. The product is used without further purification in the next step: TLC (hexane-EtOAc 2:1) Rf=0.30; ESIMS [M+H+NH₃]⁺=0.581.

### f) (R)-5-{(S)-1-tert-butoxycarbonylamino-2-[4-(6-methyl-pyridin-2-ylamino)-3-pentyl-phenyl]ethyl}-3-[1-(3-tert-butyl-phenyl)-cyclopropyl]-oxazolidin-2-one

A solution of ((S)-2-(3-pentyl-4-amino-phenyl)-1-{(R)-3-[1-(3-tert-butyl-phenyl)-cyclopropyl]-2-oxo-oxazolidin-5-yl}-ethyl)-carbamic acid tert-butyl ester (56 mg, 0.10 mmol), sodium tert-butoxide (12.5 mg, 0.13 mmol), 2-dicyclohexylphosphino-2′-(N,N-dimethylamino)biphenyl (5 mg, 0.012 mmol) and Pd₂(dba)₃ (6 mg, 0.005 mmol) in dioxane (3 ml) is degassed with argon for 5 min. 2-Chloro-6-methylpyridine is added and the solution heated under argon at 100° C. for 3 h. The solution is diluted with EtOAc, washed with saturated NaHCO₃ solution and brine, dried over Na₂SO₄, filtered and evaporated. The compound is purified with a PLC plate (Merck) 20×20 cm, silica gel 60 F₂₅₄, 1 mm (hexane-EtOAc 2:1) and eluted with EtOAc: TLC (hexane-EtOAc 2:1) Rf=0.33; ESIMS [M+H]⁺=655.

### g) (2R,3S)-3-Amino-1-[1-(3-tert-butyl-phenyl)-cyclopropylamino]-4-[4-(6-methyl-pyridin-2-ylamino)-3-pentyl-phenyl]-butan-2-ol

(R)-5-{(S)-1-tert-butoxycarbonylamino-2-[4-(6-methyl-pyridin-2-ylamino)-3-pentyl-phenyl]-ethyl}-3-[1-(3-tert-butyl-phenyl)-cyclopropyl]-oxazolidin-2-one (54 mg, 0.08 mmol) is dissolved in THF (3.0 ml). KOTMS (53 mg, 0.4 mmol) is added in one portion and the reaction mixture stirred at 130° C. in the microwave for 10 min. The reaction mixture is quenched by the addition of 0.4N HCl in dioxane solution (1 ml, 0.4 mmol) and concentrated. The crude product is used further without purification: ESIMS [M−H]⁺=529.

### h) N-{(1S,2R)-2-Hydroxy-3-[1-(3-tert-butyl-phenyl)-cyclopropylamino]-1-[4-(6-methyl-pyridin-2-ylamino)-3-pentyl-benzyl]-propyl}-acetamide

To a solution of (2R,3S)-3-Amino-1-[1-(3-tert-butyl-phenyl)-cyclopropylamino]-4-[4-(6-methyl-pyridin-2-ylamino)-3-pentyl-phenyl]-butan-2-ol (42 mg, 0.08 mmol) is added NEt₃ (56 μl). At 0° C. is added dropwise 0.1N Ac₂O in DCM (400 μl, 0.5 equivalent). After 45 min the reaction mixture is evaporated and purified using a PLC plate (Merck) 20×20 cm, silica gel 60 F₂₅₄, 1 mm (EtOAc:MeOH 10:1) and eluted with EtOAc:MeOH 10:1: TLC (EtOAc:MeOH 10:1) Rf=0.49; HPLC Rt_{D}=13.43 min; ESIMS [M+H]⁺=571.

### Examples 42 to 45

The compounds listed in Table 3 can be prepared in a manner analogous to that described in Example 7.

TABLE 3

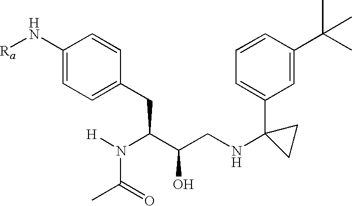

| Example | Rₐ | HPLC | ESIMS |
|---|---|---|---|
| 42 | 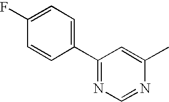 | Rt_{B} = 3.68 min | [M − H]⁺ = 582 |
| 43 | 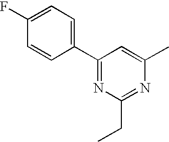 | Rt_{B} = 3.65 min | [M − H]⁺ = 610 |
| 44 | 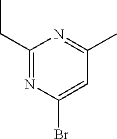 | Rt_{B} = 3.96 min | [M − H]⁺ = 596 |
| 45 | 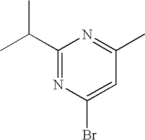 | Rt_{B} = 4.05 min | [M − H]⁺ = 609 |

### Example 46

### N-{(1S,2R)-3-[1-(3-tert-Butyl-phenyl)-cyclopropylamino]-2-hydroxy-1-[4-(6-phenyl-pyrimidin-4-ylamino)-benzyl]-propyl}-2-methoxy-acetamide

### a) (R)-5-{(S)-1-Amino-2-[4-(6-phenyl-pyrimidin-4-ylamino)-phenyl]-ethyl}-3-[1-(3-tert-butyl-phenyl)-cyclopropyl]-oxazolidin-2-one

The title compound can be prepared in a manner analogous to that described in Example 12a, starting from (R)-5-[(S)-1-amino-2-(4-amino-phenyl)-ethyl]-3-[1-(3-tert-butyl-phenyl)-cyclopropyl]-oxazolidin-2-one and 4-chloro-6-phenyl-pyrimidine: HPLC Rt_{E}=1.42 min; ESIMS [M+H]⁺=548.

### b) (2R,3S)-3-Amino-1-[1-(3-tert-butyl-phenyl)-cyclopropylamino]-4-[4-(6-phenyl-pyrimidin-4-ylamino)-phenyl]-butan-2-ol

The title compound can be prepared in a manner analogous to that described in Example 12b: HPLC Rt₈=3.52 min; ESIMS [M+H]⁺=522.

### c) N-{(1S,2R)-3-[1-(3-tert-Butyl-phenyl)-cyclopropylamino]-2-hydroxy-1-[4-(6-phenyl-pyrimidin-4-ylamino)-benzyl]-propyl}-2-methoxy-acetamide

(2R,3S)-3-Amino-1-[1-(3-tert-butyl-phenyl)-cyclopropylamino]-4-[4-(6-phenyl-pyrimidin-4-ylamino)-phenyl]-butan-2-ol (147 mg, 0.28 mmol) and DIPEA (221 μl, 1.27 mmol) are dissolved in THF. The solution is stirred for 15 min. N-(3-dimethylaminopropyl)-N′-ethyl-carbodiimide hydrochloride (60.6 mg, 0.310 mmol) and methoxyacetic acid (22.34 μl, 0.28 mmol) are added, and the mixture is stirred at RT for 3 h. The reaction mixture is quenched with water and extracted with EtOAc. The organic layer is washed with brine and dried with MgSO₄. The crude product is purified by silica gel chromatography using DCM/MeOH (98:2) to give the pure title compound: HPLC Rt_{B}=3.65 min; ESIMS [M−H]⁺=594.

### Examples 47 to 49

The compounds listed in Table 4 can be prepared in a manner analogous to that described in Example 46.

TABLE 4

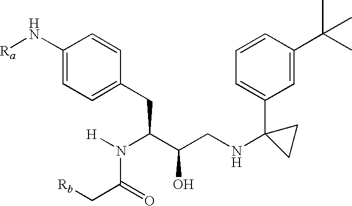

| Example | Rₐ | R_{b} | HPLC | ESIMS |
|---|---|---|---|---|
| 47 | 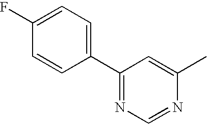 | OCH₃ | Rt_{B} = 3.70 min | [M − H]⁺ = 612 |
| 48 | 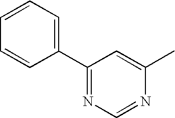 | F | Rt_{B} = 3.63 min | [M − H]⁺ = 582 |
| 49 | 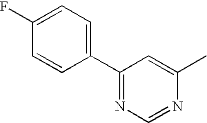 | F | Rt_{B} = 3.70 min | [M − H]⁺ = 600 |

### Example 50

### N-{(1S,2R)-3-[1-(3-tert-Butyl-phenyl)-cyclopropylamino]-2-hydroxy-1-[4-(6-methyl-4-phenyl-pyridin-2-ylamino)-benzyl]-propyl}-2-methoxy-acetamide

### a) {(S)-1-{(R)-3-[1-(3-tert-Butyl-phenyl)-cyclopropyl]-2-oxo-oxazolidin-5-yl}-2-[4-(6-methyl-4-phenyl-pyridin-2-ylamino)-phenyl]-ethyl}-carbamic acid tert-butyl ester

The title compound can be prepared in a manner analogous to that described in Example 9a, starting from ((S)-2-(4-amino-phenyl)-1-{(R)-3-[1-(3-tert-butyl-phenyl)-cyclopropyl]-2-oxo-oxazolidin-5-yl}-ethyl)-carbamic acid tert-butyl ester and 2-chloro-6-methyl-4-phenyl-pyridine: HPLC Rt_{B}=4.47 min; ESIMS [M−H]⁺=661.

### b) (R)-5-{(S)-1-Amino-2-[4-(6-methyl-4-phenyl-pyridin-2-ylamino)-phenyl]-ethyl}-3-[1-(3-tert-butyl-phenyl)-cyclopropyl]-oxazolidin-2-one

{(S)-1-{(R)-3-[1-(3-tert-Butyl-phenyl)-cyclopropyl]-2-oxo-oxazolidin-5-yl}-2-[4-(6-methyl-4-phenyl-pyridin-2-ylamino)-phenyl]-ethyl}-carbamic acid tert-butyl ester (3538 mg, 0.53 mmol) is dissolved in DCM (1.67 ml). The solution is cooled to 0° C. After the addition of TFA (1.67 ml), the mixture is stirred for 10 min at 0° C. and then for 3 h at RT. The reaction mixture is quenched with a 2 M sodium carbonate solution and extracted with DCM. The organic layer is washed with brine and dried with MgSO₄. The crude product is used without further purification [HPLC Rt_{B}=3.82 min; ESIMS [M−H]⁺=562].

### c) (2R,3S)-3-Amino-1-[1-(3-tert-butyl-phenyl)-cyclopropylamino]-4-[4-(6-methyl-4-phenyl-pyridin-2-ylamino)-phenyl]-butan-2-ol

The title compound is prepared in a manner analogous to that described in Example 12b [HPLC Rt_{B}=3.60 min; ESIMS [M−H]⁺=535].

### d) N-{(1S,2R)-3-[1-(3-tert-Butyl-phenyl)-cyclopropylamino]-2-hydroxy-1-[4-(6-methyl-4-phenyl-pyridin-2-ylamino)-benzyl]-propyl}-2-methoxy-acetamide

The title compound is prepared in a manner analogous to that described in Example 46c [HPLC Rt_{B}=3.70 min; ESIMS [M−H]⁺=607].

### Example 51

### N-{(1S,2R)-3-[1-(3-tert-Butyl-phenyl)-cyclopropylamino]-2-hydroxy-1-[4-(6-methyl-4-phenyl-pyridin-2-ylamino)-benzyl]-propyl}-2-fluoro-acetamide

The title compound is prepared in a manner analogous to that described in Example 50 [HPLC Rt_{B}=3.70 min; ESIMS [M−H]⁺=595].

### Example 52

### N-{(1S,2R)-3-[1-(3-tert-Butyl-phenyl)-cyclopropylamino]-2-hydroxy-1-[4-(2-isopropyl-6-methyl-pyrimidin-4-ylamino)-3-pentyl-benzyl]-propyl}-acetamide

The title compound is prepared in a manner analogous to that described in Example 41: TLC (EtOAc:MeOH 10:1) Rf=0.43; HPLC Rt_{D}=12.71 min; ESIMS [m+H]⁺=614.

### Example 53

### N-{(1S,2R)-3-[1-(3-tert-Butyl-phenyl)-cyclopropylamino]-2-hydroxy-1-[4-(2-isopropyl-6-methyl-pyrimidin-4-ylamino)-3-propoxy-benzyl]-propyl}-acetamide

### a) (4-Nitro-3-propoxy-phenyl)-methanol

4-Nitro-3-propoxy-benzoic acid (4.15 g, 18.4 mmol) is dissolved in THF (50 ml). NaBH₄ (1.09 g, 27.6 mmol) is slowly added and the solution is stirred for 5 min. A solution of boron trifluoride diethyletherate (1.48 ml, 11.97 mmol) in THF (25 ml) is added to the reaction mixture. The resulting solution is heated to reflux for 2 h. The reaction mixture is cooled to 0° C. and quenched with water and diluted with ether and 2N NaOH. The ether layer is washed with brine, dried with MgSO₄ and concentrated. The crude product is used without further purification [HPLC Rt_{B}=3.99 min; ESIMS [M−H]⁺=212].

### b) 4-Bromomethyl-1-nitro-2-propoxy-benzene

(4-Nitro-3-propoxy-phenyl)-methanol (3.90 g, 18.46 mmol) and triphenylphosphine (5.33 g, 20.31 mmol) are dissolved in ACN (90 ml). The solution is stirred for 10 min at RT. CBr₄ (6.75 g, 20.31 mmol) is added and the resulting mixture is stirred for 20 h. The solvent is removed and the residue is purified by silica gel chromatography using hexane/DCM (4:1) to give the title compound: ¹H-NMR (360 MHz, CDCl₃) 7.80 (d, 1H), 7.10 (s, 1H), 7.90-7.00 (d, 1H), 4.45 (s, 2H), 4.15 (t, 2H), 1.85 (m, 2H), 1.05 (t, 3H).

### c) (2R,5S)-2-Isopropyl-3,6-dimethoxy-5-(4-nitro-3-propoxy-benzyl)-2,5-dihydro-pyrazine

(R)-2-Isopropyl-3,6-dimethoxy-2,5-dihydro-pyrazine (1.97 ml, 11 mmol) is dissolved in THF (20 ml) and cooled to −75° C. A solution of n-BuLi (6.9 ml 1.6 M in hexane) is slowly added and the resulting solution is stirred for 10 min. The solution is added to a slurry of CuCN (492 mg, 5.5 mmol) and LiCl (238 mg, 5.5 mmol) in THF (25 ml) at −20° C. and is then cooled to −75° C. 4-Bromomethyl-1-nitro-2-propoxy-benzene (1.37 g, 5 mmol) in THF (5 ml) is added to the reaction mixture which is stirred for 1 h at −75° C. and 2 h at −20° C. The reaction is quenched with a saturated solution of NH₄Cl (25 ml) and stirred for 30 min. The solution is extracted with EtOAc. The organic layer is washed with brine and dried with MgSO₄. The residue is purified by silica gel chromatography using DCM/hexane (7:3) to give the title compound [HPLC Rt_{B}=3.73 min; ESIMS [M−H]⁺=378].

### d) (S)-2-Amino-3-(4-nitro-3-propoxy-phenyl)-propionic acid methyl ester

(2R,5S)-2-Isopropyl-3,6-dimethoxy-5-(4-nitro-3-propoxy-benzyl)-2,5-dihydro-pyrazine (1.8 g, 4.77 mmol) is stirred for 8 h at RT in a 0.25N HCl solution (38 ml). THF (40 ml) is added and the clear solution is stirred for 2.5 h. The THF is evaporated and the water phase is extracted with ether. The organic layer is washed with a 0.25N HCl solution. The aqueous phase is treated with a saturated NaHCO₃ solution to obtain a pH of 9, extracted with EtOAc and dried with MgSO₄ and concentrated. The residue is purified by silica gel chromatography using DCM/MeOH (99:1) to give the title compound. ¹H-NMR (360 MHz, CDCl₃) 7.70 (d, 1H), 6.90 (s, 1H), 6.80 (d, 1H), 4.05 (t, 1H), 3.75 (m, 1H), 3.70 (s, 3H), 3.15 (dd, 1H), 2.85 (dd, 1H), 1.90 (m, 2H), 1.50 (s, 1H), 1.05 (t, 3H); ESIMS [M−H]⁺=283].

### e) (S)-2-tert-Butoxycarbonylamino-3-(4-nitro-3-propoxy-phenyl)-propionic acid methyl ester

NaHCO₃ (655 mg, 7.7 mmol) is added to a solution of (S)-2-amino-3-(4-nitro-3-propoxy-phenyl)-propionic acid methyl ester (1.1 g, 3.89 mmol) in THF (15 ml) and water (20 ml). The resulting mixture is stirred for two minutes and a solution of BOC₂O (1.02 g, 4.67 mmol) in THF (5 ml) is added. The combined solution is stirred for 3.5 h at RT. The THF is removed by evaporation and the remaining water phase is extracted with EtOAc. The organic layer is washed with a saturated NaHCO₃ solution, brine and dried with MgSO₄. The residue obtained after evaporation is purified by silica gel chromatography using DCM/MeOH (99:1) to give the title compound. ¹H-NMR (360 MHz, CDCl₃) 7.70 (d, 1H), 6.90 (s, 1H), 6.80 (d, 1H), 5.05 (m, 1H), 4.65 (m, 1H), 4.05 (t, 2H), 3.75 (s, 3H), 2.95-3.15 (dd, 2H), 1.90 (m, 2H), 1.45 (s, 9H), 1.05 (t, 3H).

### f) (S)-2-tert-Butoxycarbonylamino-3-(4-nitro-3-propoxy-phenyl)-propionic acid

NaOH (45 ml, 1N) is added to a solution of (S)-2-tert-butoxycarbonylamino-3-(4-nitro-3-propoxy-phenyl)-propionic acid methyl ester (11.4 g, 45 mmol) in MeOH (70 ml). The solution is stirred at RT for 3 h. 1N HCl (75 ml) and water (150 ml) is added. The product falls out of solution and is filtered off. The title compound is obtained after recrystallization from MeOH/water. ¹H-NMR (360 MHz, CDCl₃) 7.70 (d, 1H), 6.90 (s, 1H), 6.80 (d, 1H), 5.00 (m, 1H), 4.65 (m, 1H), 4.05 (m, 2H), 3.00-3.25 (m, 2H), 1.85 (m, 2H), 1.45 (s, 9H), 1.05 (t, 3H); ESIMS [M−H]⁻=367.

### g) (S)-2-tert-Butoxycarbonylamino-3-(4-nitro-3-propoxy-phenyl)-propionic acid 4-nitro-phenyl ester

(S)-2-tert-Butoxycarbonylamino-3-(4-nitro-3-propoxy-phenyl)-propionic acid (3.3 g, 8.95 mmol) and 4-nitrophenol (1.25 g, 8.95 mmol) are dissolved in THF (16 ml) and cooled to 0° C.

A solution of N,N′-dicyclohexylcarbodiimide (1.87 g, 8.95 mmol) in THF (4 ml) is added, and the mixture is stirred at 0° C. for 3 h and 16 h at RT. The suspension is filtered and the filtrate is diluted with EtOAc. The solution is washed with a saturated K₂CO₃ solution, brine and dried with MgSO₄. The residue obtained after evaporation is purified by silica gel chromatography using DCM/MeOH (99:1) to give the title compound: ¹H-NMR (360 MHz, CDCl₃) 8.30 (d, 2H), 7.70 (d, 1H), 7.15 (d, 2H), 6.90 (s, 1H), 6.80 (d, 1H), 5.05 (m, 1H), 4.80 (m, 1H), 4.65 (m, 1H), 4.00 (m, 2H), 3.20-3.35 (m, 2H), 1.85 (m, 2H), 1.40 (s, 9H), 1.05 (t, 3H).

### h) [(S)-2-(4-Nitro-3-propoxy-phenyl)-1-(S)-oxiranyl-ethyl]-carbamic acid tert-butyl ester

1 M solution of potassium t-butoxide in THF (5.6 ml) is added to a solution of trimethyl-sulfoxonium iodide (1.7 g, 7.52 mmol) in THF (6 ml). The suspension is stirred for 2 h at 70° C. and cooled to 0° C. (S)-2-tert-Butoxycarbonylamino-3-(4-nitro-3-propoxy-phenyl)-propionic acid 4-nitro-phenyl ester (940 mg, 1.92 mmol) is dissolved in THF (4 ml) and added to the suspension. The resulting mixture is stirred at RT for 1 h and is then quenched with a saturated NaHCO₃ solution. The mixture is diluted with EtOAc. The organic layer is washed with saturated NaHCO₃, brine, dried with MgSO₄ and concentrated. The resulting sulfoxonium derivative (506 mg, 1.14 mmol) is dissolved in THF (8 ml) and cooled to 0° C. LiBr is added (100 mg, 1.14 mmol) and the resulting suspension is stirred for 10 min. Methanesulfonic acid (74.1 μl, 1.14 mmol) is added and the mixture is stirred for another 10 min and then heated for 2 h at 65° C. The reaction mixture is then quenched with a saturated NaHCO₃ solution. The mixture is diluted with EtOAc. The organic layer is washed with saturated NaHCO₃, brine, dried with MgSO₄ and concentrated. The crude product is used without further purification. NaBH₄ (79.6 mg, 2.03 mmol) is added to a cooled solution (0° C.) of [(S)-3-bromo-1-(4-nitro-3-propoxy-benzyl)-2-oxo-propyl]-carbamic acid tert-butyl ester (973 mg, 2.03 mmol) in THF (5 ml) and EtOH (10 ml). The mixture is stirred at 0° C. for 30 min and at RT for 20 h. The solvents are evaporated and the residue is treated with a saturated NaHCO₃ solution and EtOAc. The organic layer is washed with saturated NaHCO₃, brine, dried with MgSO₄ and concentrated. The residue obtained after evaporation is purified by silica gel chromatography using hexane/EtOAc (7:3) to give the title compound. ¹H-NMR (360 MHz, CDCl₃) 8.2 (d, 1H), 7.70 (d, 1H), 6.90 (s, 1H), 6.80 (d, 1H), 4.00 (m, 2H), 3.70 (m, 1H), 2.70-3.10 (m, 5H), 1.85 (m, 2H), 1.45 (s, 9H), 1.05 (t, 3H); ESIMS [M−H]⁻=365.

### i) [(1S,2R)-3-[1-(3-tert-Butyl-phenyl)-cyclopropylamino]-2-hydroxy-1-(4-nitro-3-propoxy-benzyl)-propyl]-carbamic acid tert-butyl ester

The title compound is prepared in a manner analogous to that described in Example 7a, starting from [(S)-2-(4-nitro-3-propoxy-phenyl)-1-(S)-oxiranyl-ethyl]-carbamic acid tert-butyl ester and 1-(3-tert-butyl-phenyl)-cyclopropylamine. HPLC Rt_{F}=2.82 min; ESIMS [M−H]⁺=556.

### j) (2R,3S)-3-Amino-1-[1-(3-tert-butyl-phenyl)-cyclopropylamino]-4-(4-nitro-3-propoxy-phenyl)-butan-2-ol

[(1S,2R)-3-[1-(3-tert-Butyl-phenyl)-cyclopropylamino]-2-hydroxy-1-(4-nitro-3-propoxy-benzyl)-propyl]-carbamic acid tert-butyl ester (725 mg, 1.30 mmol) is dissolved in EtOAc (15 ml). 3 N HCl in EtOAc (20 ml) is added to the cooled solution (0° C.) and the mixture is stirred at RT for 3 h. The solvents are evaporated and the residue is diluted with EtOAc. The organic layer is washed with saturated NaHCO₃, brine, dried with MgSO₄ and concentrated. The crude product is used without further purification. ESIMS [M−H]⁺=456.

### k) N-[(1S,2R)-3-[1-(3-tert-Butyl-phenyl)-cyclopropylamino]-2-hydroxy-1-(4-nitro-3-propoxy-benzyl)-propyl]-acetamide

The title compound is prepared in a manner analogous to that described in Example 7f. HPLC Rt_{B}=4.08 min; ESIMS [M−H]⁺=498.

### l) N-{(1S,2R)-1-(4-Amino-3-propoxy-benzyl)-3-[1-(3-tert-butyl-phenyl)-cyclo-propyl-amino]-2-hydroxy-propyl}-acetamide

The title compound is prepared in a manner analogous to that described in Example 4d. HPLC Rt_{B}=3.47 min; ESIMS [M−H]⁺=468.

### m) N-{(1S,2R)-3-[1-(3-tert-Butyl-phenyl)-cyclopropylamino]-2-hydroxy-1-[4-(2-isopropyl-6-methyl-pyrimidin-4-ylamino)-3-propoxy-benzyl]-propyl}-acetamide

The title compound is prepared in a manner analogous to that described in Example 4e. HPLC Rt_{F}=2.17 min; ESIMS [M−H]⁺=602.

### Example 54

### N-((1S,2R)-3-[1-(3-tert-Butyl-phenyl)-cyclopropylamino]-1-{4-[6-(4-fluoro-phenyl)-pyrimidin-4-ylamino]-3-propoxy-benzyl}-2-hydroxy-propyl)-acetamide

The title compound is prepared in a manner analogous to that described in Example 53. HPLC Rt_{F}=2.91 min; ESIMS [M−H]⁺=640.

### Example 55

### N-{(1S,2R)-3-[1-(3-tert-Butyl-phenyl)-cyclopropylamino]-1-[4-(6-chloro-2-isopropyl-pyrimidin-4-ylamino)-3-propoxy-benzyl]-2-hydroxy-propyl}-acetamide

The title compound is prepared in a manner analogous to that described in Example 53. HPLC Rt_{B}=3.09 min; ESIMS [M−H]⁺=622.

1. A compound of the formula 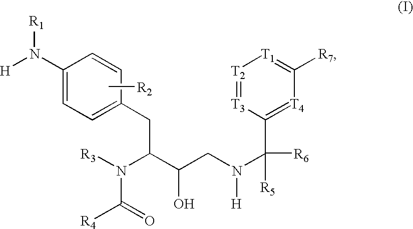
in which
R₁ is hydrogen, (C₁₋₈)alkyl, a (C₃₋₈)cycloalkyl, aryl or heteroaryl group, which (C₃₋₈)cycloalkyl, aryl or heteroaryl group is optionally substituted by 1 to 4 substituents, independently selected from the group consisting of halogen, (C₁₋₈)alkyl, halogen-substituted (C₁₋₈)alkyl, (C₁₋₈)alkoxy, (C₁₋₈)alkoxy(C₁₋₈)alkyl, (C₃₋₈)cycloalkyl and an aryl or heteroaryl group, which aryl or heteroaryl group is optionally substituted by 1 to 4 substituents, independently selected from the group consisting of halogen, (C₁₋₈)alkyl, halogen-substituted (C₁₋₈)alkyl, hydroxy, (C₁₋₈)alkoxy, (C₁₋₈)alkoxy(C₁₋₈)alkyl and (C₃₋₈)cycloalkyl, a group of the formula 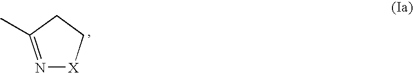
in which X is O or S, the group of the formula Ia being optionally substituted by 1 or 2 substituents, independently selected from the group consisting of halogen and (C₁₋₈)-alkyl, or a group of the formula 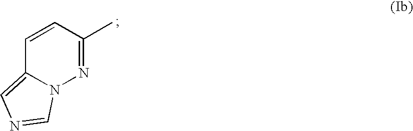
R₂ is hydrogen, halogen, (C₁₋₈)alkyl, (C₁₋₈)alkoxy, (C₁₋₈)alkoxy(C₁₋₈)alkyl, (C₁₋₈)alkylthio or a (C₃₋₈)cycloalkyl, (C₃₋₈)cycloalkyl(C₁₋₈)alkyl or (C₃₋₈)cycloalkyl(C₁₋₈)alkoxy group, in which (C₃₋₈)cycloalkyl, (C₃₋₈)cycloalkyl(C₁₋₈)alkyl or (C₃₋₈)cycloalkyl(C₁₋₈)alkoxy group the (C₃₋₈)-cycloalkyl moiety is optionally substituted by 1 to 4 substituents, independently selected from the group consisting of halogen and (C₁₋₈)alkyl;
either
R₃ is hydrogen and
R₄ is hydrogen, (C₁₋₈)alkyl, halogen-substituted (C₁₋₈)alkyl, (C₁₋₈)alkoxy(C₁₋₈)alkyl, (C₁₋₈)alkylthio(C₁₋₈)alkyl, (C₁₋₈)alkylamino(C₁₋₈)alkyl, a (C₃₋₈)cycloalkyl, aryl or heteroaryl group, which (C₃₋₈)cycloalkyl, aryl or heteroaryl group is optionally substituted by 1 to 4 substituents, independently selected from the group consisting of halogen, (C₁₋₈)alkyl, halogen-substituted (C₁₋₈)alkyl, (C₁₋₈)alkoxy, (C₁₋₈)alkoxy(C₁₋₈)alkyl, (C₃₋₈)cycloalkyl and an aryl or heteroaryl group, which aryl or heteroaryl group is optionally substituted by 1 to 4 substituents, independently selected from the group consisting of halogen, (C₁₋₈)-alkyl, halogen-substituted (C₁₋₈)alkyl, hydroxy, (C₁₋₈)alkoxy, (C₁₋₈)alkoxy(C₁₋₈)alkyl and (C₃₋₈)cycloalkyl, or a (C₃₋₈)cycloalkyl group, in which (C₃₋₈)cycloalkyl group one —CH₂-moiety is replaced by —O— and which (C₃₋₈)cycloalkyl group is optionally substituted by 1 or 2 substituents, independently selected from the group consisting of halogen and (C₁₋₈)alkyl,
or
the moiety —N(R₃)—C(═O)—R₄ is a group of the formula 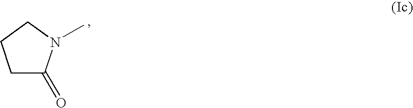
which is optionally substituted by 1 or 2 substituents, independently selected from the group consisting of halogen and (C₁₋₈)alkyl, or a group of the formula 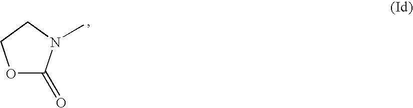
which is optionally substituted by 1 or 2 substituents, independently selected from the group consisting of halogen and (C₁₋₈)alkyl;
either
R₅ is hydrogen, (C₁₋₈)alkyl, (C₁₋₈)alkoxy(C₁₋₈)alkyl or halogen-substituted (C₁₋₈)alkyl and
R₆ is hydrogen or (C₁₋₈)alkyl
or
R₅ and R₆ together are, together with the carbon atom, to which they are attached, a (C₃₋₈)-cycloalkyl group, which (C₃₋₈)cycloalkyl group is unsubstituted or substituted by 1 to 4 substituents, independently selected from the group consisting of halogen and (C₁₋₈)alkyl;
R₇ is (C₁₋₈)alkyl, (C₃₋₈)cycloalkyl(C₁₋₈)alkyl or halogen-substituted (C₁₋₈)alkyl;
T₁ is CR₈, N, O, S or a bond;
R₈ is hydrogen, halogen, (C₁₋₈)alkyl, (C₁₋₈)alkoxy or halogen-substituted (C₁₋₈)alkyl;
T₂ is CR₉, N, O, S or a bond;
R₉ is hydrogen, halogen, (C₁₋₈)alkyl, (C₁₋₈)alkoxy or halogen-substituted (C₁₋₈)alkyl;
T₃ is CR₁₀, N, O, S or a bond;
R₁₀ is hydrogen, halogen, (C₁₋₈)alkyl, (C₁₋₈)alkoxy or halogen-substituted (C₁₋₈)alkyl;
T₄ is CR₁₁, N, O or S;
R₁₁ is hydrogen, halogen, (C₁₋₈)alkyl, (C₁₋₈)alkoxy or halogen-substituted (C₁₋₈)alkyl;
the number of ring atoms included in the ring comprising T₁ being 5 or 6;
the number of hetero ring atoms included in the ring comprising T₁ being 0, 1, 2 or 3;
the hetero ring atoms optionally included in the ring comprising T₁ being selected, if the number of ring atoms included in the ring comprising T₁ is 5, in such a way, that any ring oxygen atom, which is optionally present, is separated from any other ring oxygen atom, which is optionally present, by at least one ring atom different from a ring oxygen atom; and the hetero ring atoms optionally included in the ring comprising T₁ being selected, if the number of ring atoms included in the ring comprising T₁ is 6, in such a way, that any ring oxygen atom, which is optionally present, is separated from any other ring hetero atom, which is optionally present, by at least one ring carbon atom,
in free base form or in acid addition salt form.

2. A process for the preparation of a compound as defined in claim 1 of the formula I, in free base form or in acid addition salt form, comprising the steps of
a) reaction of a compound of the formula 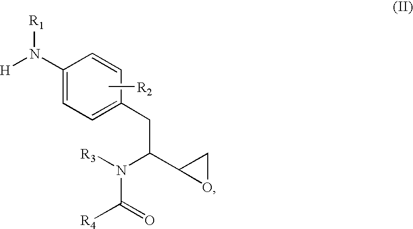
in which R₁, R₂, R₃ and R₄ are as defined for the formula I, with a compound of the formula 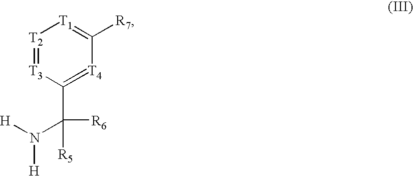
in which R₅, R₆, R₇, T₁, T₂, T₃ and T₄ are as defined for the formula I, or
b) reaction of a compound of the formula
R₁-L (IV),
in which R₁ is as defined for the formula I and L is a leaving group, with a compound of the formula 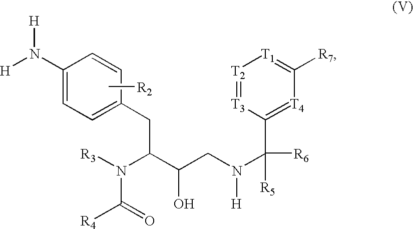
in which R₂, R₃, R₄, R₅, R₆, R₇, T₁, T₂, T₃ and T₄ are as defined for the formula I, or
c) for the preparation of a compound of the formula I, in which R₃ is hydrogen, reaction of a compound of the formula 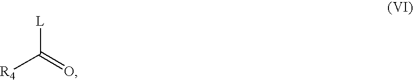
in which R₄ is as defined for the formula I and L is a leaving group, with a compound of the formula 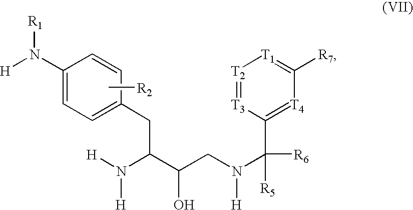
in which R₁, R₂, R₅, R₆, R₇, T₁, T₂, T₃ and T₄ are as defined for the formula I, or
d) for the preparation of a compound of the formula I, in which the moiety —N(R₃)—C(═O)—R₄ is 2-oxopyrrolidin-1-yl, intramolecular cyclisation of a compound of the formula 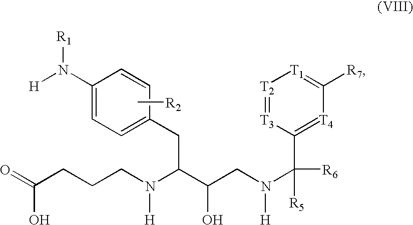
in which R₁, R₂, R₅, R₆, R₇, T₁, T₂, T₃ and T₄ are as defined for the formula I, or
in each case optionally followed by reduction, oxidation or other functionalisation of the resulting compound and/or by cleavage of any protecting group(s) optionally present, and of recovering the so obtainable compound of the formula I in free base form or in acid addition salt form.

3. A compound according to claim 1, in free base form or in pharmaceutically acceptable acid addition salt form, for use as a medicament.

4. A compound according to claim 1, in free base form or in pharmaceutically acceptable acid addition salt form, for use in the treatment of neurological or vascular disorders related to beta-amyloid generation and/or aggregation.

5. A pharmaceutical composition comprising a compound as claimed in claim 1, in free base form or in pharmaceutically acceptable acid addition salt form, as active ingredient and a pharmaceutical carrier or diluent.

6. The use of a compound as claimed in claim 1, in free base form or in pharmaceutically acceptable acid addition salt form, as a medicament for the treatment of neurological or vascular disorders related to beta-amyloid generation and/or aggregation.

7. The use of a compound as claimed in claim 1, in free base form or in pharmaceutically acceptable acid addition salt form, for the manufacture of a medicament for the treatment of neurological or vascular disorders related to beta-amyloid generation and/or aggregation.

8. A method for the treatment of neurological or vascular disorders related to beta-amyloid generation and/or aggregation in a subject in need of such treatment, which comprises administering to such subject a therapeutically effective amount of a compound as claimed in claim 1, in free base form or in pharmaceutically acceptable acid addition salt form.

9. A combination comprising a therapeutically effective amount of a compound as claimed in claim 1, in free base form or in pharmaceutically acceptable acid addition salt form, and a second drug substance, for simultaneous or sequential administration.
